SEQUENCE LISTING (2 sequences)

SEQ ID NO: 1 - DNA, 2953 bp, Homo Sapiens
atatagagacgtttccgcctcctgcttgaaactaacccctctttttctccaaaggagtgcttgtggagatcggatcttttctccagcaattgggggaaagaaggctttttctctgaattcgcttagtgtaaccagcggcgtatattttttaggcgccttttcgaaaacctagtagttaatattcatttgtttaaatcttattttatttttaagctcaaactgcttaagaataccttaattccttaaagtgaaataattttttgcaaaggggtttcctcgatttggagctttttttttcttccaccgtcatttctaactcttaaaaccaactcagttccatcatggtgatgttcaagaagatcaagtcttttgaggtggtctttaacgaccctgaaaaggtgtacggcagtggcgagaaggtggctggccgggtgatagtggaggtgtgtgaagttactcgtgtcaaagccgttaggatcctggcttgcggagtggctaaagtgctttggatgcagggatcccagcagtgcaaacagacttcggagtacctgcgctatgaagacacgcttcttctggaagaccagccaacaggtgagaatgagatggtgatcatgagacctggaaacaaatatgagtacaagttcggctttgagcttcctcaggggcctctgggaacatccttcaaaggaaaatatgggtgtgtagactactgggtgaaggcttttcttgaccgcccgagccagccaactcaagagacaaagaaaaactttgaagtagtggatctggtggatgtcaatacccctgatttaatggcacctgtgtctgctaaaaaagaaaagaaagtttcctgcatgttcattcctgatgggcgggtgtctgtctctgctcgaattgacagaaaaggattctgtgaaggtgatgagatttccatccatgctgactttgagaatacatgttcccgaattgtggtccccaaagctgccattgtggcccgccacacttaccttgccaatggccagaccaaggtgctgactcagaagttgtcatcagtcagaggcaatcatattatctcagggacatgcgcatcatggcgtggcaagagccttcgggttcagaagatcaggccttctatcctgggctgcaacatccttcgagttgaatattccttactgatctatgttagcgttcctggatccaagaaggtcatccttgacctgcccctggtaattggcagcagatcaggtctaagcagcagaacatccagcatggccagccgaaccagctctgagatgagttgggtagatctgaacatccctgataccccagaagctcctccctgctatatggatgtcattcctgaagatcaccgattggagagcccaaccactcctctgctagatgacatggatggctctcaagacagccctatctttatgtatgcccctgagttcaagttcatgccaccaccgacttatactgaggtggatccctgcatcctcaacaacaatgtgcagtgagcatgtggaagaaaagaagcagctttacctacttgtttctttttgtctctcttcctggacactcactttttcagagactcaacagtctctgcaatggagtgtgggtccaccttagcctctgacttcctaatgtaggaggtggtcagcaggcaatctcctgggccttaaaggatgcggactcatcctcagccagcgcccatgttgtgatacaggggtgtttgttggatgggtttaaaaataactagaaaaactcaggcccatccattttctcagatctccttgaaaattgaggccttttcgatagtttcgggtcaggtaaaaatggcctcctggcgtaagcttttcaaggttttttggaggctttttgtaaattgtgataggaactttggaccttgaacttatgtatcatgtggagaagagccaatttaacaaactaggaagatgaaaagggaaattgtggccaaaactttgggaaaaggaggttcttaaaatcagtgtttcccctttgtgcacttgtagaaaaaaaagaaaaaccttctagagctgatttgatggacaatggagagagctttccctgtgattataaaaaaggaagctagctgctctacggtcatctttgcttaagagtatactttaacctggcttttaaagcagtagtaactgccccaccaaaggtcttaaaagccatttttggagcctattgcactgtgttctcctactgcaaatattttcatatgggaggatggttttctcttcatgtaagtccttggaattgattctaaggtgatgttcttagcactttaattcctgtcaaattttttgttctccccttctgccatcttaaatgtaagctgaaactggtctactgtgtctctagggttaagccaaaagacaaaaaaaattttactacttttgagattgccccaatgtacagaattatataattctaacgcttaaatcatgtgaaagggttgctgctgtcagccttgcccactgtgacttcaaacccaaggaggaactcttgatcaagatgccgaaccctgtgttcagaacctccaaatactgccatgagaaactagagggcaggtcttcataaaagccctttgaacccccttcctgccctgtgttaggagatagggatattggcccctcactgcagctgccagcacttggtcagtcactctcagccatagcactttgttcactgtcctgtgtcagagcactgagctccacccttttctgagagttattacagccagaaagtgtgggctgaagatggttggtttcatgtttttgtattatgtatctttttgtatggtaaagactatattttgtacttaaccagatatatttttaccccagatggggatattctttgtaaaaaatgaaaataaagtttttttaatggaaaaaaaaaaaaaaaaaaaaaaaaaaaa

SEQ ID NO: 2 - protein, 391 aa, Homo Sapiens
MVMFKKIKSFEVVFNDPEKVYGSGEKVAGRVIVEVCEVTRVKAVRILACGVAKVLWMQGSQQCKQTSEYLRYEDTLLLEDQPTGENEMVIMRPGNKYEYKFGFELPQGPLGTSFKGKYGCVDYWVKAFLDRPSQPTQETKKNFEVVDLVDVNTPDLMAPVSAKKEKKVSCMFIPDGRVSVSARIDRKGFCEGDEISIHADFENTCSRIVVPKAAIVARHTYLANGQTKVLTQKLSSVRGNHIISGTCASWRGKSLRVQKIRPSILGCNILRVEYSLLIYVSVPGSKKVILDLPLVIGSRSGLSSRTSSMASRTSSEMSWVDLNIPDTPEAPPCYMDVIPEDHRLESPTTPLLDDMDGSQDSPIFMYAPEFKFMPPPTYTEVDPCILNNNVQ

(19) United States
(12) Patent Application Publication
(10) Pub. No.: US 2013/0224222 A1
(43) Pub. Date: Aug. 29, 2013

(54) METHODS AND ASSAYS FOR TREATING OR PREVENTING OBESITY AND/OR DIABETES OR INCREASING INSULIN SENSITIVITY

(71) Applicants: Gary J. Schwartz, Bronx, NY (US); Clemence Blouet, New York, NY (US)

(72) Inventors: Gary J. Schwartz, Bronx, NY (US); Clemence Blouet, New York, NY (US)

(21) Appl. No.: 13/641,624

(22) PCT Filed: Apr. 8, 2011
(86) PCT No.: PCT/US11/00639

§ 371 Date: Nov. 16, 2012

Related U.S. Application Data
(60) Provisional application No. 61/342,844, filed on Apr. 20, 2010.

(51) Int. Cl. G01N 33/50

(52) U.S. Cl. CPC G01N 33/5044
USPC 424/172.1; 435/6.12; 514/44 A; 435/375; 435/366; 435/7.1; 435/25; 514/44 R

(57) ABSTRACT

The present invention provides methods for determining a putative agent that treats or prevents obesity and/or diabetes or increasing insulin sensitivity, the method comprising contacting cells with the putative agent and measuring thioredoxin-interacting protein (TXNIP) or thioredoxin expression or activity in the cells. The present invention also provides the agent, the pharmaceutical composition, and methods of preventing or treating obesity and/or diabetes or of increasing insulin sensitivity or glucose sensitivity, the method comprising administration of the agent that decreases the expression of TXNIP or activity of TXNIP.

### CROSS-REFERENCE TO RELATED APPLICATIONS

This application claims benefit of U.S. Provisional Application No. 61/342,844, filed Apr. 20, 2010, the contents of which are hereby incorporated by reference.

### STATEMENT OF GOVERNMENT SUPPORT

This invention was made with government support under grant numbers DK026684, DK020541, DK047208 and DK066618 awarded by the National Institutes of Health, U.S. Department of Health and Human Services. The government has certain rights in the invention.

### FIELD OF THE INVENTION

The present invention relates generally to the downregulation of thioredoxin-interacting protein.

### BACKGROUND OF THE INVENTION

Throughout this application various publications are referred to in parenthesis. Full citations for these references may be found at the end of the specification. The disclosures of these publications are hereby incorporated by reference in their entirety into the subject application to more fully describe the art to which the subject invention pertains.

Obesity and related co-morbidities, especially type II diabetes, have reached epidemic levels in adult populations worldwide, and have an increasing impact in pediatric populations as well. Mounting evidence supports a significant role for the central nervous system, particularly the hypothalamus, in modulating behavior and metabolism relevant to the onset and maintenance of diabetes and obesity. There is an urgent need to develop therapeutic strategies that target the central nervous system in treating these disorders.

Several strategies have been proposed to increase energy expenditure (burning calories) or decrease food intake in order to correct fat mass excess and insulin signaling defects that are generally associated with obesity. To date, decreased caloric intake is achieved by diet and increased expenditure by exercise. However, both these behavioral interventions fail in the vast majority of individuals due to the complex physiology involved in both pathways.

Current therapies targeting the central nervous system control of obesity primarily act on either small molecular neurotransmitter systems (serotonin, dopamine) or more recently on orphan neuropeptide receptors for the gut peptide amylin. Serotonergic and dopaminergic compounds have shown very modest effects in terms of prolonged reductions in body weight and adipodisity, while amylin trials are more promising. However, the neuroanatomical sites, receptor subtypes, and biochemical mechanisms responsible for the beneficial metabolic effects of any of these compounds remain unclear.

Nutrient excess in obesity and diabetes is emerging as a common putative cause for multiple deleterious effects across diverse cell types, responsible for a variety of metabolic dysfunctions. Recent advances support a role for the hypothalamus in regulating whole body energy homeostasis, through both detection of nutrient availability and coordination of effectors that determine nutrient intake and utilization, thus preventing cellular and whole body nutrient excess. However, the mechanisms underlying hypothalamic nutrient detection and its impact on peripheral nutrient utilization remain poorly understood.

The hypothalamus is a major center of convergence and integration of multiple nutrient-related signals important in the regulation of energy homeostasis. In response to nutrients, adiposity and gut hormones, subsets of specialized nutrient-sensitive hypothalamic neurons engage a complex set of neurochemical and neurophysiological responses to regulate behavioral and metabolic effectors of energy balance (1), glycemic control (2) and lipid metabolism (3, 4). These hypothalamic neurons are critical in determining whole body nutrient availability, utilization and partitioning, thus preventing nutrient excess, a common feature of obesity and diabetes. However, the current understanding of the mechanisms underlying hypothalamic nutrient detection and its impacts on peripheral metabolism remains incomplete.

The present invention addresses these problems by providing methods and assays for preventing or treating obesity and/or diabetes or increasing insulin sensitivity by targeting protein expression affecting energy homeostasis

### SUMMARY OF THE INVENTION

The present invention provides a method for determining a putative agent that treats or prevents obesity and/or diabetes or increases insulin sensitivity, the method comprising contacting cells with the putative agent and measuring thioredoxin-interacting protein (TXNIP) activity or expression, or thioredoxin activity or expression in the cells, wherein a decrease in TXNIP activity or expression or increase in thioredoxin activity or expression indicates that the putative agent treats or prevents obesity and/or diabetes or increases insulin sensitivity, whereas a lack of decrease in TXNIP activity or expression or of increase in thioredoxin activity or expression indicates that the putative agent does not treat or prevent obesity and/or diabetes or increase insulin sensitivity.

The present invention additionally provides an agent that treats or prevents obesity and/or diabetes or increases insulin sensitivity, the agent identified by the method comprising contacting cells with the putative agent and measuring thioredoxin-interacting protein (TXNIP) activity or expression, or thioredoxin activity or expression in the cells, wherein a decrease in TXNIP activity or expression or increase in thioredoxin activity or expression indicates that the putative agent treats or prevents obesity and/or diabetes or increases insulin sensitivity whereas a lack of decrease in TXNIP activity or expression or of increase in thioredoxin activity or expression indicates that the putative agent does not treat or prevent obesity and/or diabetes or increase insulin sensitivity.

The present invention further provides a pharmaceutical composition comprising a therapeutically effective amount of the agent in a pharmaceutically acceptable carrier.

The present invention provides a method of preventing or treating obesity and/or diabetes or of increasing insulin sensitivity in a subject, the method comprising administering to the subject a therapeutically effective amount of an agent or pharmaceutical composition thereof that decreases the activity or expression of thioredoxin-interacting protein (TXNIP).

The present invention also provides a method of increasing glucose tolerance or insulin sensitivity in cells, the method comprising administering to the cells an effective amount of an agent or pharmaceutical composition that decreases the activity or expression of thioredoxin-interacting protein (TXNIP).

The present invention provides the use of a transfection vector that decreases the activity or expression of thioredoxin-interacting protein (TXNIP) to prevent or treat obesity and/or diabetes or to increase insulin sensitivity. The present invention additionally provides the use of an agent or a pharmaceutical composition thereof that decreases the activity or expression of thioredoxin-interacting protein (TXNIP) to prevent or treat obesity and/or diabetes or to increase insulin sensitivity.

A method is provided for determining whether an agent treats or prevents obesity and/or treats or prevents diabetes or increases insulin sensitivity, the method comprising contacting a cell which expresses thioredoxin-interacting protein (TXNIP) with the agent and measuring (i) TXNIP expression or TXNIP activity in the cell, or (ii) thioredoxin expression or activity in the cell, wherein a decrease in TXNIP expression or activity relative to a predetermined level thereof, or an increase in thioredoxin expression or activity relative to a predetermined level thereof, indicates that the agent treats or prevents obesity and/or diabetes or increases insulin sensitivity, whereas a lack of decrease in TXNIP expression or activity, or a lack of increase in thioredoxin expression or activity, indicates that the agent does not treat or prevent obesity and/or diabetes or increase insulin sensitivity.

Also provided is a pharmaceutical composition comprising (1) an antibody directed against TXNIP which inhibits TXNIP expression or activity, an aptamer which inhibits TXNIP expression or activity, or a molecule which effects RNAi-mediated inhibition or siRNA inhibition of TXNIP expression or activity and (2) pharmaceutically acceptable carrier.

Also provided is a method of preventing or treating obesity and/or diabetes or of increasing insulin sensitivity in a subject, the method comprising administering to the subject a therapeutically effective amount of an agent or pharmaceutical composition comprising an agent that decreases activity of, or expression of, thioredoxin-interacting protein (TXNIP).

Also provided is a method of increasing glucose tolerance or insulin sensitivity in cells, the method comprising administering to the cells an effective amount of an agent or pharmaceutical composition comprising an agent that decreases activity of, or expression of, thioredoxin-interacting protein (TXNIP).

Also provided is use of a transfection vector that decreases the expression of, or the activity of, thioredoxin-interacting protein (TXNIP) to prevent or treat obesity and/or diabetes or to increase insulin sensitivity.

Also provided is use of an agent or a pharmaceutical composition that decreases the expression of, or the activity of, thioredoxin-interacting protein (TXNIP) to prevent or treat obesity and/or diabetes or to increase insulin sensitivity.

### BRIEF DESCRIPTION OF THE DRAWINGS

FIG. 1A-1D. TXNIP is expressed in the hypothalamus in the mouse brain. Immunohistochemistry showing TXNIP expression in (A) the arcuate, (B) the ventromedial, (C) the paraventricular and (D) the lateral nuclei of the hypothalamus. Scale bar: 50 microns.

FIG. 2A-2H. TXNIP in the MBH (medial basal hypothalamus) responds to nutritional and hormonal signals and is a target of FoxO1. (A) TXNIP mRNA expression corrected to actin. (B) TXNIP protein expression corrected to actin and (C) MBH thioredoxin activity in 24 h fasted (F) or refed (RF) mice (n=4-6). (D) MBH TXNIP protein expression corrected to actin in mouse following local MBH administration of aCSF, insulin or leptin (n=5). MBH (E) FoxO1 and (F) TXNIP protein expression corrected to actin in fasted (F) or refed (RF) wild type and FoxO1, heterozygous mice (n=5-6). MBH (G) FoxO1 and (H) TXNIP protein expression corrected to actin in mice after an MBH injection of a GFP, FOXO ADA or FOXO D256 adenovirus (n=5). All data are means+/−SEM. *:P,0.05, **:P,0.01 vs. control.

FIG. 3A-3O. MBH TXNIP overexpression increases body weight and body fat, decreases energy expenditure and impairs glycemic control. (A) Body weight, (B) daily food intake, (C) body composition, (D) total spontaneous physical activity, (E) respiratory quotient, (F) oxygen comsumption and (G) brown fat temperature in mice fed a high fat diet following MBH injection of C247S hTXNIP, LacZ (controls) or hTXNIP lentivirus (injection on day 0, n=6-10). (H) Blood glucose during an ip. insulin sensitivity test in mice fed a HFD 4 weeks following the viral injections (n=10). (I) Blood glucose and (J) plasma insulin during an OGTT in mice fed a HFD 3 weeks following the viral injections (n=10). (K) Blood glucose, (L) plasma insulin, (m) plasma triglycerides, (n) plasma non-esterified free fatty acids and (O) plasma beta-hydroxybutyrate in 24 h fasted (F, n=10) or 4 h refed (RF, n=10) mice fed a high-fat diet and injected with C247S hTXNIP, LacZ or hTXNIP lentivirus into the MBH. All data are means+/−SEM. *:P<0.05, **:P<0.001 vs. controls.

FIG. 4A-4J. TXNIP overexpression in the MBH reduces AgRP/NPY expression, induces oxidative stress and impairs leptin and insulin action. (A) AgRP, NPY and POMC mRNA corrected to actin in the MBH of mice infected with the C247S hTXNIP, LacZ and hTXNIP lentivirus (n=10). (B) Oxidized (GSSG) and total (GSH) glutathione concentrations, (C) UCP2, NRF1, NRF2, SOD1 and SOD2 mRNA expression corrected to actin and (D) ROS production in the MBH of mice infected with C247S hTXNIP, LacZ or hTXNIP lentivirus in the MBH (n=5). (E) Mediobasal hypothalamic insulin-induced Akt Ser 319 phosphorylation and (F) leptin-induced STAT3 Y705 phosphorylation in mice expressing hTXNIP or C247S hTXNIP in the MBH (n=5). (G) Leptin-induced anorexia and body weight change in mice expressing hTXNIP or C247S hTXNIP in the MBH following an intra-MBH leptin (L, 150 ng) or aCSF (A) injection (n=4). (H) Recording samples showing the response of NPY neurons to exogenous application of leptin (50 nM) in current clamp mode. (I) Pooled data from 20 neurons showing changes in the membrane potential in response to leptin. (J) PTEN expression in non-reducing and reducing conditions in mice expressing hTXNIP or C247S hTXNIP in the MBH (n=6). All data are means+/−SEM. *:P<0.05, **:P<0.01, ***:P<0.001 vs. controls.

FIG. 5A-5G. MBH TXNIP overexpression impairs sympathetic activity to white and brown fat and adipose tissue metabolism. (A) Plasma NEFA and brown fat temperature change following an ip. administration of CL, a (33-receptor agonist, (B) cold sensitivity during a 2 h cold challenge at 4° C., (C) body weight loss during a 24 h fast and food intake during the subsequent 4 h refeeding period in mice injected with C247S or hTXNIP lentivirus into the MBH (n=5). (D) Brown fat PGC1α, UCP1 and β3 adrenergic receptor mRNA expression corrected to actin, (E) visceral fat β3 AMPkα Thr172 phosphorylation, ACC Ser79 phosphorylation and HSL Ser563 phosphorylation, and (G) visceral fat f4/80 and TNF mRNA expression corrected to actin in mice injected with C247S hTXNIP, LacZ or hTXNIP lentivirus into the MBH and after an overnight fast (F, n=5) or a 4 h refeeding (RF, n=5). All data are means+/−SEM. *:P<0.05, **:P<0.01, ***:P<0.001 vs. controls.

FIG. 6A-6G. MBH TXNIP is a therapeutic target in the treatment of obesity and insulin resistance. MBH TXNIP protein expression corrected to actin in overnight fasted (F) or 4 h refed (RF) refed C57/B16 mice or ob/ob mice (A), NONcNZ10/LtJ obese and diabetic mice (B), and STZ treated mice (C). Body weight (D), blood sugar in response to an ip. insulin challenge (E), and blood glucose (F) and plasma insulin (G) in response to an oral glucose challenge in HFD-fed mice expressing TXNIP shRNA or a control shRNA in the MBH. All values are means+/−SEM. *:P<0.05, **:P<0.01, ***:P<0.001 vs. control.

FIG. 7: Endo Ra, Rd, and percentage suppression of hepatic glucose production (HGP) during a euglycemic hyperinsulinemic clamp (D) in HFD-fed mice expressing TXNIP shRNA or a control shRNA in the MBH (n=5-8)

### DETAILED DESCRIPTION OF THE INVENTION

The present invention provides a method for determining a putative agent that treats or prevents obesity and/or diabetes or increases insulin sensitivity, the method comprising contacting cells with the putative agent and measuring thioredoxin-interacting protein (TXNIP) activity or expression, or thioredoxin expression expression in the cells, wherein a decrease in TXNIP activity or expression or increase in thioredoxin activity or expression indicates that the putative agent treats or prevents obesity and/or diabetes or increases insulin sensitivity, whereas a lack of decrease in TXNIP activity or expression or of increase in thioredoxin activity or expression indicates that the putative agent does not treat or prevent obesity and/or diabetes or increase insulin sensitivity.

Thioredoxin-interacting protein (TXNIP) is an endogenous negative regulator of thioredoxin (5), which in humans, is encoded by the TXNIP gene. TXNIP is one of the main ubiquitously expressed thiol-reducing non-enzymatic antioxidants that inhibits the reducing activity of thioredoxin by interacting with its catalytic active center (6). Thioredoxin is a protein which, in humans, is encoded by the TXN gene. Thioredoxin acts as an antioxidant by facilitating the reduction of other proteins by cysteine thiol-disulfide exchange. Thioredoxins are found in nearly all known organisms, from bacteria to mammals. In the hypothalamus, TXNIP regulates adipose tissue metabolism, fuel partitioning and glucose homeostasis. TXNIP upregulation occurs during fasting in normal physiological conditions and is downregulated upon refeeding. Nutritional and viral manipulation of TXNIP expression affects hypothalamic neurons involved in control of white and brown adipose tissue function and gene expression, energy expenditure, food intake, temperature regulation, arousal and locomotor behavior.

Obesity and diabetes are adverse metabolic consequences of high fat diets. Insulin resistance results in increased release of insulin from the pancreas in response to an increase in blood glucose level. Chronic elevated insulin may result in diabetes, metabolic syndrome, heart disease, or other diseases or disorders. Increasing insulin sensitivity can prevent the onset of more severe or debilitating health conditions.

Determination of a putative agent that treats or prevents obesity and/or diabetes or increases insulin sensitivity, can be done in vitro or in vivo. If in vitro, the determination can be performed on any cell expressing TXNIP. Preferably, the cells are mammalian, for example, from a rodent or human. Preferably, the cells are from the hypothalamus. If in vivo, determination can be performed on any subject. Preferably, the subject is mammalian, for example, a rodent or human. Preferably, the cells in vitro and the subject in vivo are in the fasted state prior to contacting the cells with the putative agent or administering the putative agent to the subject.

In vitro, the level of TXNIP or thioredoxin activity or expression in a cell contacted by the putative agent can be determined by any method known in the art. For example, a Western blot can be performed for expression levels. In vivo, glucose tolerance or insulin sensitivity can be measured by any method known in the art, for example, an oral glucose tolerance test or an insulin sensitivity test (IST). The method may further comprise comparing the level of TXNIP or thioredoxin activity or expression in a cell contacted by the putative agent or the glucose tolerance or insulin sensitivity of a subject administered the putative agent to at least one control. In one embodiment, in vitro, the level of TXNIP or thioredoxin activity or expression in a cell contacted by the putative agent can be compared to at least one of the following controls: (1) the TXNIP or thioredoxin activity or expression in cells not contacted by the putative agent or, when the cells contacted with the putative agent are in the fasting state, (2) the TXNIP or thioredoxin activity or expression in cells in the fed state contacted by the putative agent. In one embodiment, in vivo, the glucose tolerance or insulin sensitivity of a subject administered the putative agent can be compared to at least one of the following controls: (1) the glucose tolerance or insulin sensitivity of the same subject before administration of the putative agent or (2) the glucose tolerance or insulin sensitivity of a control subject that is not administered the putative agent. Preferably, the control subject is physiologically similar to the subject being administered the putative agent.

The putative agent may be administered to the subject by any method known in the art. Preferably, the putative agent is administered directly to the subject's hypothalamus.

The present invention additionally provides an agent that treats or prevents obesity and/or diabetes or increases insulin sensitivity, the agent identified by the method comprising contacting cells with the putative agent and measuring thioredoxin-interacting protein (TXNIP) or thioredoxin activity or expression in the cells, wherein a decrease in TXNIP activity or expression or increase in thioredoxin activity or expression indicates that the putative agent treats or prevents obesity and/or diabetes or increases insulin sensitivity whereas a lack of decrease in TXNIP activity or expression or of increase in thioredoxin activity or expression indicates that the putative agent does not treat or prevent obesity and/or diabetes or increase insulin sensitivity.

The putative agent in the present invention can be any chemical or biological agent for example, a chemical, small compound, polypeptide, protein, protein fragment, peptide mimetic, or aptamer. An aptamer may be a single stranded oligonucleotide or oligonucleotide analog that binds to a particular target molecule, such as a protein. Alternatively, the aptamer may be a protein aptamer, which consists of a variable peptide loop attached at both ends to a protein scaffold that interferes with protein interaction. A peptide mimetic is a short peptide, which mimics the sequence of a protein of interest. Preferably, the putative agent is membrane-permeable. Preferably, the putative agent can cross the blood-brain barrier. Alternatively, the putative agent may be a transfection vector comprising cDNA for a transcription or translation inhibitor of TXNIP. Preferably, the vector is specific to the hypothalamus or to cell types within the hypothalamus.

Also provided is a method for determining whether an agent treats or prevents obesity and/or treats or prevents diabetes or increases insulin sensitivity, the method comprising contacting a cell which expresses thioredoxin-interacting protein (TXNIP) with the agent and measuring (i) TXNIP expression or TXNIP activity in the cell, or (ii) thioredoxin expression or activity in the cell, wherein a decrease in TXNIP expression or activity relative to a predetermined level thereof, or an increase in thioredoxin expression or activity relative to a predetermined level thereof, indicates that the agent treats or prevents obesity and/or diabetes or increases insulin sensitivity, whereas a lack of decrease in TXNIP expression or activity, or a lack of increase in thioredoxin expression or activity, indicates that the agent does not treat or prevent obesity and/or diabetes or increase insulin sensitivity.

In an embodiment, the method is for determining whether an agent treats obesity. In an embodiment, the method is for determining whether an agent prevents obesity. In an embodiment, the method is for determining whether an agent treats diabetes. In an embodiment, the method is for determining whether an agent prevents diabetes. In an embodiment, the method is for determining whether an agent increases insulin sensitivity.

In an embodiment, the method comprises measuring TXNIP expression or activity in the cell. In an embodiment, the method comprises measuring thioredoxin expression or activity in the cell. In an embodiment, the cell is in a fasted state. In an embodiment, the predetermined level of expression or activity is determined from the cell in the absence of the agent. In an embodiment, the predetermined level of expression or activity is determined from the cell in a fed state and contacted with the agent. In an embodiment, the cells are mammalian. In an embodiment, the cells are human. In an embodiment, the cell is a hypothalamic cell or is derived from a hypothalamus. In an embodiment, the method is for determining if the agent treats or prevents obesity.

Also provided is a pharmaceutical composition comprising (1) an antibody directed against TXNIP which inhibits TXNIP expression or activity, an aptamer which inhibits TXNIP expression or activity, or a molecule which effects RNAi-mediated inhibition or siRNA inhibition of TXNIP expression or activity and (2) pharmaceutically acceptable carrier.

The present invention further provides a pharmaceutical composition comprising a therapeutically effective amount of an agent which downregulates the activity or expression of TXNIP in a pharmaceutically acceptable carrier. The pharmaceutical composition may comprise the agent in a pharmaceutically acceptable carrier. Alternatively, the pharmaceutical composition may consist essentially of the agent in a pharmaceutically acceptable carrier. Yet alternatively, the pharmaceutical composition may consist of the agent in a pharmaceutically acceptable carrier.

The pharmaceutically acceptable carrier must be compatible with the agent, and not deleterious to the subject. Examples of acceptable pharmaceutical carriers include carboxymethylcellulose, crystalline cellulose, glycerin, gum arabic, lactose, magnesium stearate, methylcellulose, powders, saline, sodium alginate, sucrose, starch, talc, and water, among others. Formulations of the pharmaceutical composition may conveniently be presented in unit dosage and may be prepared by any method known in the pharmaceutical art. For example, the agent may be brought into association with a carrier or diluent, as a suspension or solution. Optionally, one or more accessory ingredients, such as buffers, flavoring agents, surface-active ingredients, and the like, may also be added. The choice of carriers will depend on the method of administration. The pharmaceutical composition can be formulated for administration by any method known in the art, including but not limited to, intravenously and intracranially. Preferably, the agent or pharmaceutical composition thereof is administered directly to hypothalamus cells. Alternatively, the agent or pharmaceutical composition thereof may be administered in a method that results in the preferential downregulation of TXNIP in hypothalamus cells.

The pharmaceutical composition would be useful for administering the agent to a subject to prevent or treat obesity and/or diabetes or increase insulin sensitivity. The agent is provided in amounts effective to prevent or treat obesity and/or diabetes or increase insulin sensitivity. These amounts may be readily determined by one of a variety of standard pharmacological approaches. In one embodiment, the agent is the sole active pharmaceutical ingredient in the formulation or composition. In another embodiment, there may be a number of active pharmaceutical ingredients in the formulation or composition aside from the agent. In this embodiment, the other active pharmaceutical ingredients in the formulation or composition must be compatible with the agent.

Also provided is a method of preventing or treating obesity and/or diabetes or of increasing insulin sensitivity in a subject, the method comprising administering to the subject a therapeutically effective amount of an agent or pharmaceutical composition comprising an agent that decreases activity of, or expression of, thioredoxin-interacting protein (TXNIP).

In an embodiment, the agent or pharmaceutical composition decreases the activity of TXNIP. In an embodiment, the subject is human. In an embodiment, the agent is, or the pharmaceutical composition comprises, an antibody directed against TXNIP which inhibits TXNIP expression or activity, or an siRNA which inhibits TXNIP expression or activity. In an embodiment, the agent or pharmaceutical composition is administered to the subject's central nervous system in a manner effective to reach the subject's hypothalamus.

Also provided is a method of increasing glucose tolerance or insulin sensitivity in cells, the method comprising administering to the cells an effective amount of an agent or pharmaceutical composition comprising an agent that decreases activity of, or expression of, thioredoxin-interacting protein (TXNIP).

In an embodiment, the agent or pharmaceutical composition decreases the activity of TXNIP. In an embodiment, the cells are human. In an embodiment, the agent is, wherein the agent is, or the pharmaceutical composition comprises, an antibody directed against TXNIP which inhibits TXNIP expression or activity, or an siRNA which inhibits TXNIP expression or activity. In an embodiment, the cells are hypothalamic cells.

In an embodiment of the methods, the agent or pharmaceutical composition comprises a transfection vector comprising cDNA coding for a TXNIP transcription inhibitor or a TXNIP translation inhibitor. In an embodiment of the methods, the agent or pharmaceutical composition comprises leptin, insulin, or a mimetic thereof. In an embodiment of the methods, the agent or pharmaceutical composition comprises a STAT3 inhibitor, a PI3 kinase activator, or a mimetic thereof. In an embodiment of the methods, the agent or pharmaceutical composition comprises FoxO1.

Also provided is use of a transfection vector that decreases the expression of, or the activity of, thioredoxin-interacting protein (TXNIP) to prevent or treat obesity and/or diabetes or to increase insulin sensitivity. In an embodiment, the transfection vector is specific to hypothalamic cells.

Also provided is use of an agent or a pharmaceutical composition that decreases the expression of, or the activity of, thioredoxin-interacting protein (TXNIP) to prevent or treat obesity and/or diabetes or to increase insulin sensitivity.

In an embodiment, the agent or pharmaceutical composition is administered directly to hypothalamus cells in a mammal. In an embodiment, the agent or pharmaceutical composition comprises an antibody directed against TXNIP which inhibits TXNIP expression or activity, or an siRNA which inhibits TXNIP expression or activity

Preventing obesity, or any grammatical equivalent thereof, as used herein, means administering the agent or pharmaceutical composition in a manner and amount sufficient to forestall the subject from becoming clinically obese. Treating obesity, or any grammatical equivalent thereof, as used herein, means administering the agent or pharmaceutical composition in a manner and amount sufficient to affect a clinically significant reduction in the subject's obesity. One skilled in the art can easily determine the amount and manner of administration of agent or pharmaceutical composition necessary. Obesity, or any grammatical equivalent thereof, as used herein, is characterized by the subject having a body mass index of 30.0 or greater (and thus includes the states of significant obesity, morbid obesity, super obesity, and super morbid obesity). In regard to gender, women with over 30% body fat are considered obese, and men with over 25% body fat are considered obese. The methods of treating obesity as disclosed herein are also applicable to treating an overweight state in a subject, defined as a body mass index of the subject of from 25.0 to 29.9, so as to stabilize, reduce, ameliorate or eliminate a sign or symptom of the overweight state in the subject.

Preventing diabetes, or any grammatical equivalent thereof, as used herein, means administering the agent or pharmaceutical composition in a manner and amount sufficient to forestall the subject from becoming clinically diabetic. Treating diabetes, or any grammatical equivalent thereof, as used herein, means administering the agent or pharmaceutical composition in a manner and amount sufficient to affect a clinically significant reduction in the severity of the subject's diabetes. One skilled in the art can easily determine the amount and manner of administration of agent or pharmaceutical composition necessary.

Increasing insulin sensitivity, or any grammatical equivalent thereof, as used herein, means administering the agent or pharmaceutical composition in a manner and amount sufficient to affect a clinically significant reduction in the subject's insulin resistance. The subject's insulin resistance may be measured by any method known in the art, including but not limited to, fasting insulin levels and glucose tolerance testing. One skilled in the art can easily determine the amount and manner of administration of agent or pharmaceutical composition necessary.

Preferably the subject in any of the instant methods is a mammal, such as a rodent or human.

The agent or pharmaceutical composition may be administered by any method known in the art, such as intravenously or intracranially. Preferably, the agent or pharmaceutical composition is administered directly to the subject's hypothalamus or is administered in a manner which effectively.

The present invention also provides a method of increasing glucose tolerance or insulin sensitivity in cells, the method comprising administering to the cells an effective amount of an agent or pharmaceutical composition that decreases the expression of thioredoxin-interacting protein (TXNIP).

Impaired glucose tolerance (IGT) is a pre-diabetic state of dysglycemia that is associated with insulin resistance and increased risk of cardiovascular pathology. IGT may precede type 2 diabetes mellitus by many years. IGT is also a risk factor for mortality. Increasing a glucose tolerance in cells means administering the agent or pharmaceutical composition in a manner and amount sufficient to affect a significant increase in the cell's glucose tolerance. The cell's glucose tolerance may be measured by any method known in the art, including but not limited to, glucose tolerance testing and fasting glucose tests. One skilled in the art can readily determine the amount and manner of administration of agent or pharmaceutical composition necessary.

The agent or pharmaceutical composition thereof that downregulates TXNIP expression may comprise the agent or pharmaceutical composition thereof identified by the method comprising contacting cells with the putative agent and measuring TXNIP or thioredoxin expression in the cells, wherein a decrease in TXNIP expression or increase in thioredoxin expression indicates that the putative agent treats or prevents obesity and/or diabetes or increases insulin sensitivity whereas a lack of decrease in TXNIP expression or of increase in thioredoxin expression indicates that the putative agent does not treat or prevent obesity and/or diabetes or increase insulin sensitivity.

The cells can be any cells that express TXNIP. Preferably, the cells are mammalian, such as from a rodent or human. Preferably, the cells are from a hypothalamus.

Transfection is a process of deliberately introducing heterologous nucleic acid(s) into a cell such that expression of the heterologous nucleic acid or a portion thereof occurs in the cell. When a viral method is used, the virus mediating the transfer of the genetic material is the vector. A cell is transduced when a vector introduces the heterologous nucleic acid into the cell. Certain viruses preferentially infect certain tissue- or cell-types. For example, the vector used of the transfection may be chosen to preferentially infect the hypothalamus or a particular cell type found in the hypothalamus.

The vector may be administered locally or systemically. For example, the vector may be administered locally via injection into the hypothalamus in vivo or via contacting hypothalamus cells in vitro. Systemic administration may include, for example, administering via in vivo injection into the circulatory system.

The transfection vector may comprise any genetic sequence that, upon cellular transduction, will decrease the expression or cellular retention of TXNIP or decrease the activity of TXNIP. Any transfection vector known in the art may be used. IN an embodiment, the transfection vector comprises cDNA coding for a TXNIP transcription or cDNA coding for a TXNIP translation inhibitor. Preferably, the transfection vector is specific to the hypothalamus or hypothalamus cells.

Any agent or pharmaceutical composition that downregulates TXNIP expression may be used. Examples of such agents include, but are not limited to, leptin, insulin, or mimetics thereof. Examples of downstream inhibitors and/or activators of leptin or insulin which can be used include, but are not limited to, STAT3 activators and PI3 inhibitors, or mimetics thereof. In another example, the agent that downregulates TXNIP expression may comprise the transcription factor FoxO1.

FoxO1 (forkhead box O1) is a protein in the forkhead family of transcription factors which, in humans is encoded by the FoxO1 gene. FoxO1 is active in the fasting state, is inhibited by both insulin and leptin in the MBH, contributes to hypothalamic nutrient detection, regulates TXNIP expression in a human liver cell line, and negatively regulates TXNIP expression in the MBH.

The agent or pharmaceutical composition thereof may be administered by any method known in the art. Preferably, the agent or pharmaceutical composition thereof is administered directly to the hypothalamus cells. Alternatively, the agent or pharmaceutical composition thereof may be administered in a method that results in the preferential downregulation of TXNIP in hypothalamus cells.

The present invention further provides the use of a transfection vector that decreases the expression of thioredoxin-interacting protein (TXNIP) to prevent or treat obesity and/or diabetes or to increase insulin sensitivity. The present invention additionally provides the use of an agent or a pharmaceutical composition thereof that decreases the expression of thioredoxin-interacting protein (TXNIP) to prevent or treat obesity and/or diabetes or to increase insulin sensitivity.

In an embodiment, the siRNA (small interfering RNA) as used in the methods or compositions described herein comprises a portion which is complementary to an mRNA sequence encoded by NCBI Reference Sequence: NM_{—}006472.3, and the siRNA is effective to inhibit expression of thioredoxin interacting protein (TXNIP). In an embodiment, the siRNA comprises a double-stranded portion (duplex). In an embodiment, the siRNA is 20-25 nucleotides in length. In an embodiment the siRNA comprises a 19-21 core RNA duplex with a one or 2 nucleotide 3′ overhang on, independently, either one or both strands. The siRNA can be 5′ phosphorylated or not and may be modified with any of the known modifications in the art to improve efficacy and/or resistance to nuclease degradation. In an embodiment the siRNA can be administered such that it is transfected into one or more cells.

In an embodiment, the mRNA encoding TXNIP has the sequence:

| (SEQ ID NO: 1) | |
|---|---|
| 1 | atatagagac gtttccgcct cctgcttgaa actaacccct ctttttctcc aaaggagtgc |
| 61 | ttgtggagat cggatctttt ctccagcaat tgggggaaag aaggcttttt ctctgaattc |
| 121 | gcttagtgta accagcggcg tatatttttt aggcgccttt tcgaaaacct agtagttaat |
| 181 | attcatttgt ttaaatctta ttttattttt aagctcaaac tgcttaagaa taccttaatt |
| 241 | ccttaaagtg aaataatttt ttgcaaaggg gtttcctcga tttggagctt tttttttctt |
| 301 | ccaccgtcat ttctaactct taaaaccaac tcagttccat catggtgatg ttcaagaaga |
| 361 | tcaagtcttt tgaggtggtc tttaacgacc ctgaaaaggt gtacggcagt ggcgagaagg |
| 421 | tggctggccg ggtgatagtg gaggtgtgtg aagttactcg tgtcaaagcc gttaggatcc |
| 481 | tggcttgcgg agtggctaaa gtgctttgga tgcagggatc ccagcagtgc aaacagactt |
| 541 | cggagtacct gcgctatgaa gacacgcttc ttctggaaga ccagccaaca ggtgagaatg |
| 601 | agatggtgat catgagacct ggaaacaaat atgagtacaa gttcggcttt gagcttcctc |
| 661 | aggggcctct gggaacatcc ttcaaaggaa aatatgggtg tgtagactac tgggtgaagg |
| 721 | cttttcttga ccgcccgagc cagccaactc aagagacaaa gaaaaacttt gaagtagtgg |
| 781 | atctggtgga tgtcaatacc cctgatttaa tggcacctgt gtctgctaaa aaagaaaaga |
| 841 | aagtttcctg catgttcatt cctgatgggc gggtgtctgt ctctgctcga attgacagaa |
| 901 | aaggattctg tgaaggtgat gagatttcca tccatgctga ctttgagaat acatgttccc |
| 961 | gaattgtggt ccccaaagct gccattgtgg cccgccacac ttaccttgcc aatggccaga |
| 1021 | ccaaggtgct gactcagaag ttgtcatcag tcagaggcaa tcatattatc tcagggacat |
| 1081 | gcgcatcatg gcgtggcaag agccttcggg ttcagaagat caggccttct atcctgggct |
| 1141 | gcaacatcct tcgagttgaa tattccttac tgatctatgt tagcgttcct ggatccaaga |
| 1201 | aggtcatcct tgacctgccc ctggtaattg gcagcagatc aggtctaagc agcagaacat |
| 1261 | ccagcatggc cagccgaacc agctctgaga tgagttgggt agatctgaac atccctgata |
| 1321 | ccccagaagc tcctccctgc tatatggatg tcattcctga agatcaccga ttggagagcc |
| 1381 | caaccactcc tctgctagat gacatggatg gctctcaaga cagccctatc tttatgtatg |
| 1441 | cccctgagtt caagttcatg ccaccaccga cttatactga ggtggatccc tgcatcctca |
| 1501 | acaacaatgt gcagtgagca tgtggaagaa aagaagcagc tttacctact tgtttctttt |
| 1561 | tgtctctctt cctggacact cactttttca gagactcaac agtctctgca atggagtgtg |
| 1621 | ggtccacctt agcctctgac ttcctaatgt aggaggtggt cagcaggcaa tctcctgggc |
| 1681 | cttaaaggat gcggactcat cctcagccag cgcccatgtt gtgatacagg ggtgtttgtt |
| 1741 | ggatgggttt aaaaataact agaaaaactc aggcccatcc attttctcag atctccttga |
| 1801 | aaattgaggc cttttcgata gtttcgggtc aggtaaaaat ggcctcctgg cgtaagcttt |
| 1861 | tcaaggtttt ttggaggctt tttgtaaatt gtgataggaa ctttggacct tgaacttatg |
| 1921 | tatcatgtgg agaagagcca atttaacaaa ctaggaagat gaaaagggaa attgtggcca |
| 1981 | aaactttggg aaaaggaggt tcttaaaatc agtgtttccc ctttgtgcac ttgtagaaaa |
| 2041 | aaaagaaaaa ccttctagag ctgatttgat ggacaatgga gagagctttc cctgtgatta |
| 2101 | taaaaaagga agctagctgc tctacggtca tctttgctta agagtatact ttaacctggc |
| 2161 | ttttaaagca gtagtaactg ccccaccaaa ggtcttaaaa gccatttttg gagcctattg |
| 2221 | cactgtgttc tcctactgca aatattttca tatgggagga tggttttctc ttcatgtaag |
| 2281 | tccttggaat tgattctaag gtgatgttct tagcacttta attcctgtca aattttttgt |
| 2341 | tctccccttc tgccatctta aatgtaagct gaaactggtc tactgtgtct ctagggttaa |
| 2401 | gccaaaagac aaaaaaaatt ttactacttt tgagattgcc ccaatgtaca gaattatata |
| 2461 | attctaacgc ttaaatcatg tgaaagggtt gctgctgtca gccttgccca ctgtgacttc |
| 2521 | aaacccaagg aggaactctt gatcaagatg ccgaaccctg tgttcagaac ctccaaatac |
| 2581 | tgccatgaga aactagaggg caggtcttca taaaagccct ttgaaccccc ttcctgccct |
| 2641 | gtgttaggag atagggatat tggcccctca ctgcagctgc cagcacttgg tcagtcactc |
| 2701 | tcagccatag cactttgttc actgtcctgt gtcagagcac tgagctccac ccttttctga |
| 2761 | gagttattac agccagaaag tgtgggctga agatggttgg tttcatgttt ttgtattatg |
| 2821 | tatctttttg tatggtaaag actatatttt gtacttaacc agatatattt ttaccccaga |
| 2881 | tggggatatt ctttgtaaaa aatgaaaata aagttttttt aatggaaaaa aaaaaaaaaa |
| 2941 | aaaaaaaaaa aaa |

In one embodiment, a siRNA of the invention comprises a double-stranded RNA wherein one strand of the double-stranded RNA is 80, 85, 90, 95 or 100% complementary to a portion of an RNA transcript of a gene encoding TXNIP. In another embodiment, a siRNA of the invention comprises a double-stranded RNA wherein one strand of the RNA comprises a portion having a sequence the same as a portion of 18-25 consecutive nucleotides of an RNA transcript of a gene encoding TXNIP. In yet another embodiment, a siRNA of the invention comprises a double-stranded RNA wherein both strands of RNA are connected by a non-nucleotide linker. Alternately, a siRNA of the invention comprises a double-stranded RNA wherein both strands of RNA are connected by a nucleotide linker, such as a loop or stem loop structure.

In one embodiment, a single strand component of a siRNA of the invention is from 14 to 50 nucleotides in length. In another embodiment, a single strand component of a siRNA of the invention is 14, 15, 16, 17, 18, 19, 20, 21, 22, 23, 24, 25, 26, 27, or 28 nucleotides in length. In yet another embodiment, a single strand component of a siRNA of the invention is 21 nucleotides in length. In yet another embodiment, a single strand component of a siRNA of the invention is 22 nucleotides in length. In yet another embodiment, a single strand component of a siRNA of the invention is 23 nucleotides in length. In one embodiment, a siRNA of the invention is from 28 to 56 nucleotides in length.

In another embodiment, a siRNA of the invention is 40, 41, 42, 43, 44, 45, 46, 47, 48, 49, 50, 51, or 52 nucleotides in length. In yet another embodiment, a siRNA of the invention is 46 nucleotides in length.

In another embodiment, an siRNA of the invention comprises at least one 2′-sugar modification. In another embodiment, an siRNA of the invention comprises at least one nucleic acid base modification. In another embodiment, an siRNA of the invention comprises at least one phosphate backbone modification.

In an embodiment, the TXNIP has the sequence set forth in NCBI Reference Sequence: NP_{—}006463.3. In an embodiment the TXNIP has the sequence:

| (SEQ ID NO: 2) | |
|---|---|
| 1 | MVMFKKIKSF EVVFNDPEKV YGSGEKVAGR VIVEVCEVTR VKAVRILACG VAKVLWMQGS |
| 61 | QQCKQTSEYL RYEDTLLLED QPTGENEMVI MRPGNKYEYK FGFELPQGPL GTSFKGKYGC |
| 121 | VDYWVKAFLD RPSQPTQETK KNFEVVDLVD VNTPDLMAPV SAKKEKKVSC MFIPDGRVSV |
| 181 | SARIDRKGFC EGDEISIHAD FENTCSRIVV PKAAIVARHT YLANGQTKVL TQKLSSVRGN |
| 241 | HIISGTCASW RGKSLRVQKI RPSILGCNIL RVEYSLLIYV SVPGSKKVIL DLPLVIGSRS |
| 301 | GLSSRTSSMA SRTSSEMSWV DLNIPDTPEA PPCYMDVIPE DHRLESPTTP LLDDMDGSQD |
| 361 | SPIFMYAPEF KFMPPPTYTE VDPCILNNNV Q |

As used herein an “aptamer” is a single-stranded oligonucleotide or oligonucleotide analog that binds to a particular target molecule, such as a TXNIP, or to a nucleic acid encoding a TXNIP, and inhibits the function or expression thereof, as appropriate. Alternatively, an aptamer may be a protein aptamer which consists of a variable peptide loop attached at both ends to a protein scaffold that interferes with TXNIP protein interactions.

As used herein “a predetermined level” of activity or expression is an amount of activity or expression, respectively, determined from a control. The control may be the same cells/system as used in the method or assay in the absence of the agent, or in the presence of the agent and in a fed state, or any other suitable control. The predetermined level may be determined from a population of cells of the same type or from subjects of the same type. Selection of the control is a standard procedure for one skilled in the art and the predetermined level is selected based on the state, e.g. resting, to which the agent is being determined as changing the activity or expression of relative to.

All combinations of the various elements described herein are within the scope of the invention unless otherwise indicated herein or otherwise clearly contradicted by context.

Where a numerical range is provided herein, it is understood that all numerical subsets of that range, and all the individual integers contained therein, are provided as part of the invention. Thus, an siRNA which is from 18 to 25 nucleotides in length includes the subset of siRNAs which are 18 to 22 nucleotides in length, the subset of siRNAs which are 20 to 25 nucleotides in length etc. as well as a siRNA which is 18 nucleotides in length, a siRNA which is 19 nucleotides in length, a siRNA which is 20 nucleotides in length, etc.up to and including a siRNA which is 25 nucleotides in length.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### 1. Methods and Materials

### Animals

Male C57/B16, B6.FVB-Tg(Npy-hrGFP)1Lowl/J (or NPY-GFP, expressing a humanized renilla GFP under control of the mouse neuropeptide Y), ob/ob on a C57/B16 background, B6.Cg-Tg(Mc4r-MAPT/GFP*)21Rck/J (or Mc4R-GFP, expressing a tau (MAPT)-sapphire GFP under the transcriptional control of the mouse melanocortin 4 receptor promoter), and NONcNZO10/LtJ mice were obtained at 10 weeks of age from Jackson Laboratories (Bar Harbor, Me.). POMC-CRE Z/eGFP and LepR-Rosa mice were obtained as previously described (47). C3H congenic TXNIP-deficient Hcb19 mice harboring a naturally occurring nonsense mutation in the TXNIP gene and control C3H/DiSnA (C3H) mice have been previously described (8). All animals were housed in individual cages and maintained in a temperature-controlled room under a standard light/dark cycle with ad libitum access to water and standard chow unless specifically indicated.

### Stereotaxic Surgery and Viral Injections

Stereotaxic surgery was performed under ketamine/xylazine anaesthesia. Mice were bilaterally injected with adenovirus or lentivirus particles (2×10¹⁰ pfu/ml for adenovirus particles and 1×10⁹ pfu/ml for lentivirus particles, 500 nl/side over a 10-min period) expressing either human Txnip (hTXNIP), C247S hTXNIP, FoxO1-ADA, FoxO1 D256, GFP or LACZ in the MBH (coordinates from bregma: A/P—1.1 mm, D/V—5.9 mm). hTXNIP and C247S hTXNIP plasmids were a gift from Richard Lee and were packaged into lentiviruses by System Bioscience (Mountain View, Calif.). FoxO1-ADA, FoxO1 D256 and GFP adenoviruses were a gift from Domenico Accili (Columbia University). LACZ lentivirus was purchased from Genecure (Norcross, Ga.). In some cases a chronic bilateral intrahypothalamic cannula (Plastics One Inc.) was implanted. All mice were sacrificed by decapitation and hypothalamic nuclei were dissected as previously described (14). Successful adenovirus or lentivirus administration in the MBH was confirmed by immunoblot analysis with TXNIP or FoxO1 antibody. All experimental protocols were approved by the Institute for Animal Studies of the Albert Einstein College of Medicine.

### Metabolic Phenotyping

One week prior to lentivirus injection, mice were adapted to individual feeding chambers (Med Associates) equipped with 20-mg pellets dispensers and fed ad libitum with a standard chow diet or a high fat diet (Biosery precision pellets F05524, 15.8 kJ/g and Biosery precision pellets F06294, 22.8 kJ/g, respectively). Food intake was monitored continuously from 4 days before to 20-25 days after virus administration and body weight was assessed daily. Meal patterns were determined as previously described (48). Body composition was determined by magnetic resonance spectroscopy using an ECHO MRS instrument (Echo Medical Systems). To determine energy expenditure, mice were adapted to individual metabolic chambers. Metabolic measurements (oxygen consumption, carbon dioxide production, food intake and locomotor activity) were obtained continuously using a CLAMS (Columbus Instruments) open-circuit indirect calorimetry system for 7 consecutive days. Glucose tolerance was assessed with a 1 g.kg⁻¹ BW oral glucose challenge after a 6 h daytime fast with tail blood sampling. Insulin sensitivity was assessed using a 0.75 U ip. insulin challenge after a 6 h daytime fast with tail blood sampling.

### Streptozotocin Treatment

Mice received a single ip. injection of streptozotocin (Promega, 0.175 g/kg body weight in 0.1 mol/L citrate). Blood glucose levels were measured daily and animals were sacrificed for TXNIP expression experiments 4 days later.

### CL Challenge

After blood collection for basal measurements, mice received an ip. injection of 1 mg/kg BW CL316243 (Sigma), a β3-adrenergic agonist. Blood was collected 15 min later for plasma NEFA measurement and plasma NEFA change over that time period was calculated. Brown fat temperature was monitored as described below, over the 60 min following the CL injection, and brown fat temperature change over that time period was calculated.

### Brown Fat Temperature Monitoring

Mice were implanted with radiofrequency impedance temperature probes (MiniMitter) under the intrascapular brown fat pad, under isoflurane anesthesia, and allowed a 1-week recovery. Brown fat temperature was recorded using MiniMitter ER-4000 receivers. For the cold challenge experiment, mice were exposed for 2 h to 4° C. and brown fat temperature was recorded continuously.

### MBH Leptin, Insulin and Glucose Infusions

Overnight food deprived mice equipped with bilateral cannulae targeting the MBH received an MBH injection of aCSF, leptin (recombinant mouse leptin, R&D Systems, 150 ng in 150 nl per side over 5 min), insulin (human insulin, Actrapid, 250 μU in 100 nl per side over 5 min) or glucose (Sigma, 1 μl of 20% glucose over 4 h) and MBH extracts were collected 30 min later for signaling studies or 4 h after the beginning of the infusion to assess TXNIP expression. MBH leptin-induced STAT3 activation and AMPk inhibition, and MBH insulin-induced Akt phosphorylation were assessed as described below. Hypothalamic leptin sensitivity was assessed by measuring 24 h food intake and body weight change following a local leptin injection in the MBH (150 ng in 150 nl per side) after a 6 h fast 1 h before the onset of the dark.

### Adenovirus Functional Validation In Vitro

N41 embryonic hypothalamic cells (Cellutions Biosystems) were plated in DMEM with 4500 mg/L glucose 24 hours before transduction with hTXNIP, LACZ, TXNIP shRNA or control shRNA lentivirus. 48 h later, cells were harvested and processed for TXNIP expression as described.

### Real-Time PCR

RNA extraction and real-time PCR experiments were performed as previously described (49). Briefly, total RNA was isolated from frozen MBH wedges, white and brown adipose tissue using RNeasy kits (Quiagen), according to the manufacturer's instructions. Extracted RNA was quantified using a NanoDrop ND-1000 (Nanodrop) and RNA integrity was confirmed with Ethidium Bromide staining. Following treatment with DNase I (Invitrogen), purified RNA was used as template for first-strand cDNA synthesis using SuperScript III (Invitrogen). Quantitative real-time RT-PCR was run using LC-Fast Start DNA SYBR Green I chemistry (Roche Diagnostics) on a LightCycler 2.0 platform (Roche Diagnostics). Samples of RNA in which the reverse transcriptase was omitted and samples without cDNA were included as negative controls. Relative quantification of each transcript in comparison to β-actin was determined as previously reported (49).

### Immunoblot Analysis

White adipose tissue was homogenized in 50 mM Tris, 1 mM EGTA, 1 mM EDTA, 50 mM sodium fluoride, 10 mM β-glycerophosphate, 20 mM sodium pyrophosphate, 2 mM orthovanadate, 2 mM PMSF, and Complete phosphatase inhibitor cocktail (Roche), without Triton X-100. After low-speed centrifugation (2,600 g at 4° C.), the fat layer was removed and Triton X-100 was added to a final concentration of 1%. Hypothalamic wedges and brown adipose tissue were directly homogenized in the Triton-containing buffer. After incubation at 4° C. for 30 min, the extracts were cleared by centrifugation at 20,000 g for 15 min. Protein concentration was measured with a BCA protein quantification kit (Pierce Biotechnology). Protein extracts were run on Criterion gels (Bio-Rad) and blotted onto nitrocellulose membranes (Millipore). After blocking for 1 h at room temperature, immunoblots were incubated overnight at 4° C. in primary antibodies against FoxO1, phospho-STAT3 (Tyr705), STAT3, phospho-aAMPk (Thr 172), aAMPk, phospho-Akt (Ser473), Akt, PTEN, phospho-HSL (Ser563), HSL, phospho-ACC (Ser79), ACC (Cell Signaling Technology), TXNIP (MBL International), thioredoxin (Abeam), or β-actin (Santa Cruz Biotechnology). Blots were then incubated for 1 hour in fluorescent (Alexa Fluor 680-conjugated anti-mouse IgG, Invitrogen or IR Dye 800-conjugated goat anti-rabbit IgG, Rockland Immunochemicals) or HRP-linked (anti-rabbit or anti-mouse HRP-linked IgG, Cell Signaling Technology) secondary antibodies and proteins were detected using either the fluorescence-based Odyssey Infrared Imaging System (LI-COR Biosciences) or enhanced chemiluminescence (ECL Plus, Amersham). Quantification was performed with the gel analyze tool of the Odyssey software (http://www.licor.com/bio/odyssey/index.jsp) or the NIH Image/J software (http://rsbweb.nih.gov/ij/), respectively.

### Thioredoxin Activity Assay

Thioredoxin “insulin-reducing assay” was performed as described (50), with some modifications. MBH wedges were homogenized in 20 mM HEPES pH7.9, 100 mM KCl, 300 mM NaCl, 10 mM EDTA, 0.1% Triton 2 mM sodium orthovanadate and antiprotease cocktail (Roche). 100 μg protein in 102 μl were incubated for 15 min at 37° C. with 3 μl DTT activation buffer (50 mM HEPES pH 7.6, 1 mM EDTA, 1 mg/ml BSA, 2 mM DTT). 60 μl reaction mixture (200 μl of 1M HEPES pH 7.6, 40 μl of 0.2 m EDTA, 40 μl of NADPH 40 mg/ml and 500 μl insulin at 10 mg/ml) was then added to each sample. 40 μl of each sample was then transferred to a 96-well plate and 0.25 U thioredoxin reductase or water (negative control) was added, followed by incubation at 37° C. for 20 min. The reaction was stopped by addition of 187.5 μl stop buffer (6M guanidine HCl, 1 mM DTNB in 0.2 M Tris-HCl pH8). Absorbance at 412 nm was read against a thioredoxin standard curve.

### Glutathione Assay

Oxidized (GSSG) and total glutathione (GSH) concentrations were assessed as follows. Briefly, MBH wedges were homogenized in 5% trichloroacetic acid (TCA). For GSSG assay, 25 μl homogenate were incubated for 1 hour at room temperature with 1 μl 2-vinylpiridine (50% in ethanol) and 2 μl triethanolamine (50% in H₂O₂). For GSH assay, samples were diluted 1:5 in TCA. 10 μl of each sample was mixed with 175 μl NADPH (0.248 mg/ml in 143 mM NaH₂PO₄ and 6.3 mM Na₄EDTA, pH 7.5), 25 μl DTNB (6 mM in 143 mM NaH₂PO₄ and 6.3 mM Na₄EDTA, pH 7.5) and 40 μl H₂O₂ preheated at 37° C. 10 GSSG reductase (10 U/ml) was added and absorbance at 405 nm was read continuously for the following 20 min. GSH and GSSG levels were determined using the kinetic slope against a GSSG standard curve and standardized to the sample protein amount, determined using the Bradford assay.

### ROS Production

Intracellular ROS production assessed by measuring changes in fluorescence resulting from intracellular probe CM-H2DCFDA (Invitrogen) oxidation, as described (22). Briefly, MBH wedges were collected in 400 μl of 5 mM HEPES, pH 7.4, frozen in liquid nitrogen and rapidly thawed the day of the experiment. After incubation at 37° C. for 45 min in 400 μl CM-H₂DCFDA (8 μmol/L in 5 mM HEPES, pH 7.4) under agitation, samples were incubated for 15 min at 4° C. in 200 μl lysis buffer (0.1% SDS and 50 mM TrisHCl pH 7.4), homogenized and centrifuged at 6,000 g for 20 min at 4° C. Supernatants were collected and fluorescence was measured with a SPECTRA Fluor Plus plate reader (Tecan U.S. Inc., Durham, N.C.) at an excitation wavelength of 485 nm and an emission wavelength of 535 nm and normalized to samples' protein content.

### Tissue Collection for Immunostaining

Mice were anaesthetized using pentobarbital. Brains were perfused transcardially via a 23-gauge needle placed in the left ventricle with 100 ml of 0.1 M PBS (pH 7.4) followed by 100 ml of 4% paraformaldehyde in PBS, and the fixed brains were cryoprotected in 30% sucrose. Coronal hypothalamic sections of 25 to 35 μm thickness were prepared on a freezing microtome.

### TXNIP Immunofluorescence

Free floating sections were incubated in 0.3% hydrogen peroxide for 15 min, blocked with 10% normal goat serum (NGS) and then incubated in TXNIP antiserum (1:500 dilution, MBL International) with 0.3% Triton X-100 and 3% NGS in PBS for 48 h at 4° C. with gentle agitation. Sections were then exposed for 2 h to cy3-conjugated goat antimouse (1:200; Jackson Immunoresearch) washed in PBS, floated onto gelatinized slides and coverslipped with Vectashield (Vector Laboratories). Native GFP and cy3 fluorescence were visualized with the appropriate lasers and emission filters on a LSM 510 NLO multiphoton confocal microscope (Zeiss, Thornwood, N.Y.).

### TXNIP and GFP Double-Labeling

Free floating sections incubated for 15 min in 0.3% hydrogen peroxide and blocked 2 h in 5% normal donkey serum (NDS) before being incubated overnight at room temperature in goat anti-GFP (1:1000, Abcam) in 0.3% Triton X-100 and 3% NDS. Sections were then incubated for 2 h with 594 Alexafluor donkey anti-goat (1:1000, Invitrogen), washed in 0.1% Triton X-100 and incubated in TXNIP antiserum (1:500 dilution, MBL International) with 0.3% Triton X-100 and 3% NDS in PBS for 48 h at 4° C. with gentle agitation. Sections were then exposed for 2 h to 488 Alexafluor donkey anti-mouse (1:1000; Jackson Immunoresearch) washed in PBS, floated onto gelatinized slides and coverslipped with Vectashield.

### Image Analysis.

Images of tissue sections were digitized, and areas of interest were outlined based on cellular morphology. TXNIP-positive and GFP-positive cells within the regions of interest were quantified with automated image analysis software (NIH Image and Zeiss Axiovision 4.6 software), and counted by the imaging programs by setting minimum and maximum optical density levels. Brain regions evaluated were lateral hypothalamus (0.7-0.8 mm caudal to bregma), paraventricular hypothalamus (0.7-0.8 mm caudal to bregma), arcuate nucleus (1.8-1.9 mm caudal to bregma), dorsomedial hypothalamus (1.7-1.8 mm caudal to bregma) and the nucleus of the solitary tract at the mid-level of the area postrema (7.3-7.4 mm caudal to bregma), corresponding to the coordinates in the brain atlas of Paxinos and Franklin (51), and were based on 4 animals/group.

### Slice Preparation

Transverse brain slices were prepared from mice at postnatal age 8-9 weeks. Animals were anesthetized with a mixture of ketamine and xylazine. After decapitation, the brain was transferred into a sucrose-based solution bubbled with 95%0₂-5% CO₂ and maintained at ˜3° C. This solution contained (mM): sucrose 248; KCl 2; MgCl₂ 1; KH₂PO₄ 1.25; NaHCO₃ 26 and glucose 10. Transverse coronal brain slices (200 μM) were prepared using a Vibratome (Leica VT1000S). Slices were equilibrated with an oxygenated artificial cerebrospinal fluid (aCSF) for >1 hour prior to transfer to the recording chamber. The slices were continuously superfused with aCSF at a rate of 2 ml/min containing (in mM); NaCl 113, KCl 3, NaH₂PO₄ 1, NaHCO₃ 26, CaCl₂ 2.5, MgCl₂ 1 and glucose 5 in 95% O₂/5% CO₂ at room temperature.

### Electrophysiological Recordings

Brain slices were placed on the stage of an upright, infrared-differential interference contrast microscope (Olympus BX50WI) mounted on a Gibraltar X-Y table (Burleigh) and visualized with a 40× water immersion objective by infrared microscopy (DAGE MTI camera). Membrane potentials were recorded at 28° C. to a PC after being filtered at 2 kHz by a Multiclamp 700B and analyzed using pClamp 10 (Axon instruments, Inc). The external solution contained (in mM): NaCl 113, KCl 3, NaH₂PO₄ 1, NaHCO₃ 26, CaCl₂ 2.5, MgCl₂ 1 and glucose 5 in 95% O₂/5% CO₂. The internal solution contained (mM): Kacetate; 115; KCl 10; MgCl₂ 2; EGTA 10; HEPES 10; Na₂ATP, 2, Na₂GTP 0.5 and phosphocreatine 10. Pipette resistance ranged from 3 to 4MΩ. Membrane potential was measured before, during and after application of leptin (50 nM) in current clamp mode. After at least 5 min of stable recording, leptin was applied to the NPY neurons via bath application.

### Analytical Procedures

Blood glucose levels were determined using a Glucometer (Precision Xtra, MediSense), plasma insulin levels using ELISA (Linco Mouse Insulin Kit), plasma triglycerides, NEFA and β-hydroxybutyrate using a colorimetric assay (Sigma, Wako and Biovision, respectively). Fasted levels were assessed after an overnight food deprivation and refed levels after a 4 h refeeding period. For NEFA and triglycerides analysis, blood was collected on para-oxon ethyl (lipase inhibitor) and EDTA coated tubes, and for insulin and β-hydroxybutyrate, on EDTA coated tubes.

### Statistical Analysis

All data, presented as means±SEM, have been analyzed using GraphPad Prism 5. For all statistical tests, an a risk of 5% was used. All kinetics were analyzed using a mixed model for repeated measurements. Multiple comparisons were tested with an ANOVA and adjusted with Tukey post-tests. Single comparisons were made using one-tail Student T-tests.

### 2. Results

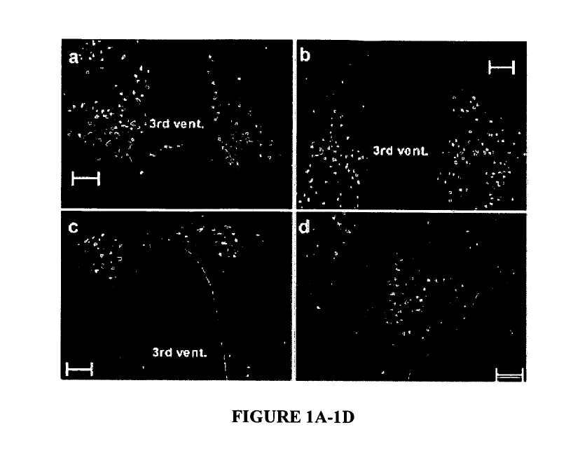

TXNIP protein was first localized in the mouse brain using immunohistochemistry. The specificity of the TXNIP antibody was confirmed by immunohistochemistry and western blot in TXNIP-deficient Hcb19 mice and their wild type littermates, C3H mice. Although TXNIP expression was absent in most of the brain, TXNIP was highly expressed in the arcuate, the ventromedial, the lateral and the paraventricular nuclei of the hypothalamus (FIG. 1A-1D). The use of transgenic animals expressing a GFP in specific neuronal populations relevant to the melanocortinergic control of energy balance revealed that TXNIP was expressed in leptin receptor (LepR), melanocortin 4 receptor (MC4R), neuropeptide Y (NPY) and proopiomelanocortin (POMC) expressing neurons. Thus, in the brain, TXNIP is expressed selectively in specific hypothalamic neuronal subpopulations involved in the control of energy homeostasis.

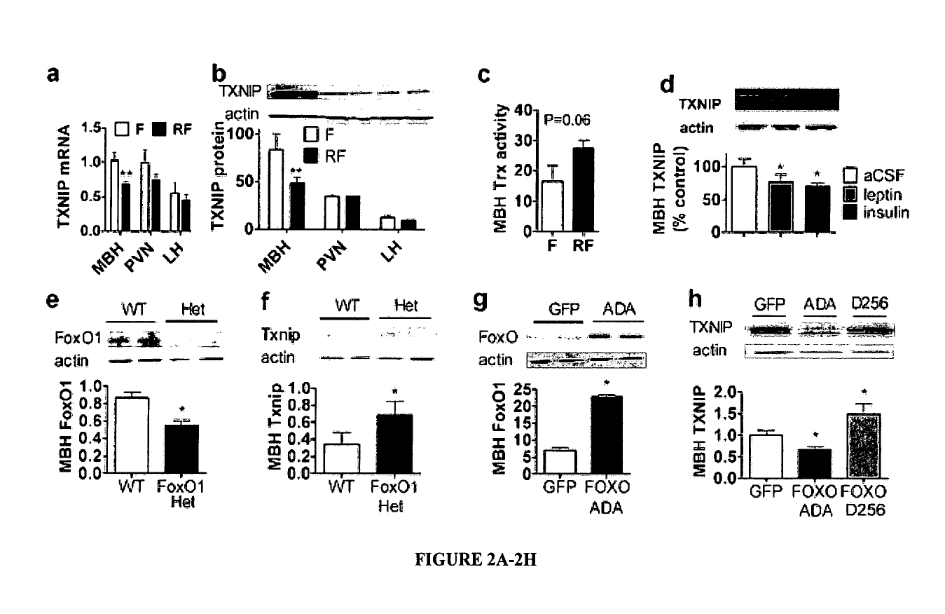

To begin to assess the physiological relevance of hypothalamic TXNIP in the regulation of energy homeostasis, it was next investigated whether changes in nutrient availability affect hypothalamic TXNIP expression. In the mediobasal hypothalamus (MBH), both TXNIP mRNA and protein expression were significantly lower in refed mice compared to fasted controls, whereas nutritional status did not significantly affect TXNIP expression in the paraventricular and lateral nuclei of the hypothalamus (FIG. 2A, 2B). Consistent with the lower MBH TXNIP expression in the refed state, thioredoxin activity tended to be higher in refed mice compared to fasted controls (FIG. 2C). Thus, hypothalamic TXNIP expression responds to changes in nutrient availability specifically in the MBH. Since fasting and refeeding induce multiple responses in various organs that could directly or indirectly affect TXNIP expression in the MBH, the effect of local, MBH parenchymal administrations insulin and leptin on MBH TXNIP expression was assessed. The volumes used for these injections (100 to 150 nl/side) were sufficiently low to ensure site specificity, as reported elsewhere (14), did not reach the paraventricular nucleus of the hypothalamus, lateral hypothalamus, or dorsomedial hypothalamus, and did not induce any changes in circulating glucose, leptin or insulin. Acute administration of both insulin and leptin decreased MBH TXNIP expression (FIG. 2D), supporting a role for these adiposity hormones in the changes in MBH TXNIP expression observed during the feeding/fasting transition.

Because Forkhead box O1 (FoxO1) is a transcription factor active in the fasting state, inhibited by both insulin and leptin in the MBH, known to contribute to hypothalamic nutrient detection (15) and has been implicated in the regulation of TXNIP expression in a human liver cell line (16), it was asked whether FoxO1 regulates TXNIP expression in the MBH. To address this question, both FoxO1 haploinsufficent mice (17) (18) and adenovectors to overactivate or downregulate FoxO1 selectively in the MBH of adult mice were used. In FoxO1 haploinsufficient mice, exhibiting a 50% decrease in FoxO1 expression in the MBH compared to wild type littermates (FIG. 2E), a 2-fold increase in TXNIP expression in the MBH was found (FIG. 2F). Consistent with this latter result, expression of a constitutively nuclear mutant of FoxO1 (FoxO1 ADA, resistant to nuclear exclusions by PI3K agonists (19)) in the MBH, achieved through a site-specific stereotaxic injection (FIG. 2G), significantly reduced TXNIP expression in the MBH (FIG. 2H); conversely, injection of an adenovirus expressing a dominant negative mutant of FoxO1 (FoxO1 D256, lacking the transactivation domain (17)), increased TXNIP expression in the MBH (FIG. 2H). Taken together, these data indicate that FoxO1 negatively regulates TXNIP expression in the MBH.

To investigate the specific role of MBH TXNIP in the regulation of energy homeostasis, the mediobasal hypothalamus was stereotaxically targeted with lentivectors expressing human TXNIP (hTXNIP), a mutant of hTXNIP (C247S hTXNIP, mutation of a single cysteine, Cys-247, that abolishes the ability of TXNIP to bind thioredoxin and inhibit thioredoxin activity (20)) or LACZ, and evaluated the effects of these manipulations on multiple behavioral and metabolic effectors of energy balance. Lentivirus functional validity was confirmed both in N41 hypothalamic cells and in MBH extracts from injected mice. MBH infection with the hTXNIP lentivirus led to a 2-fold increase in total TXNIP protein expression in the MBH, without altering TXNIP expression in the PVN or the LH and induced a 2-fold decrease in thioredoxin activity in the MBH.

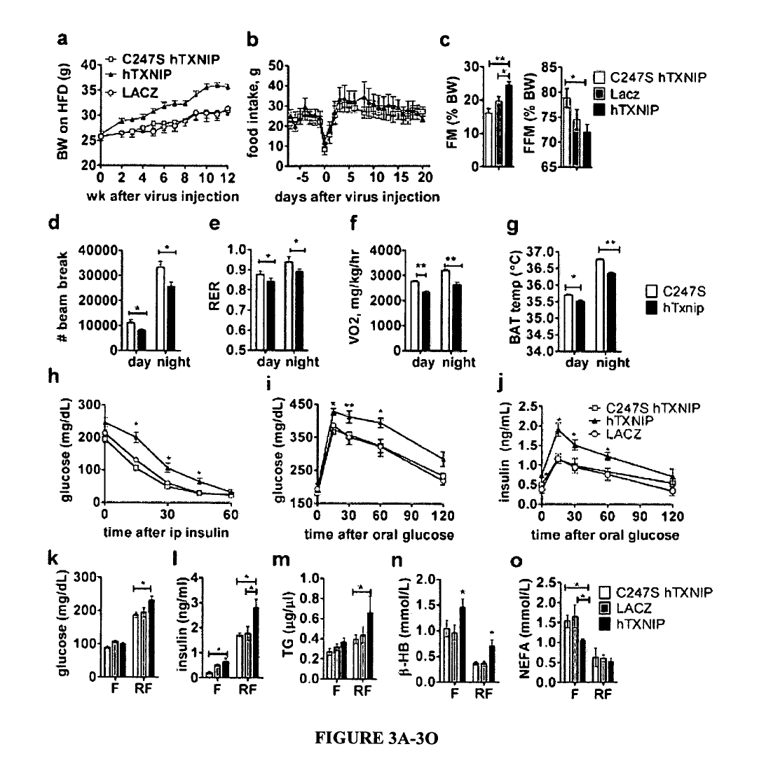

Although MBH TXNIP overexpression did not significantly affect food intake, body weight gain or body composition when mice were maintained on a normal chow diet, under high fat feeding, TXNIP overexpression in the MBH increased body weight gain, fat mass and decreased lean body mass compared to expression of both C247S hTXNIP and LACZ control vectors (FIG. 3A, 3C). These changes occurred in the absence of any effect on feeding behavior (FIG. 3B). Oxygen consumption, respiratory quotient, physical activity and brown fat temperature were decreased in MBH hTXNIP-expressing mice compared to C247S-expressing controls (FIG. 3D-3G), suggesting that lower energy expenditure in MBH hTXNIP-expressing mice accounted for their higher rate of body weight gain. MBH TXNIP overexpression also impaired glycemic control, as evidenced by the glucose intolerance, the impaired response to ip. insulin, the postprandial hyperglycemia and the fasting and postprandial hyperinsulinemia in MBH hTXNIP-expressing mice compared to both LACZ- and C247S hTXNIP-expressing controls (FIG. 3H-3L). Last, overexpression of TXNIP in the MBH resulted in hypertriglyceridemia, hyperketosis and decreased fasting levels of plasma non-esterified free fatty acid (NEFA) (FIG. 3M-30). Together, these data demonstrate the role of MBH TXNIP in the control of nutrient partitioning, fuel utilization and glucose homeostasis.

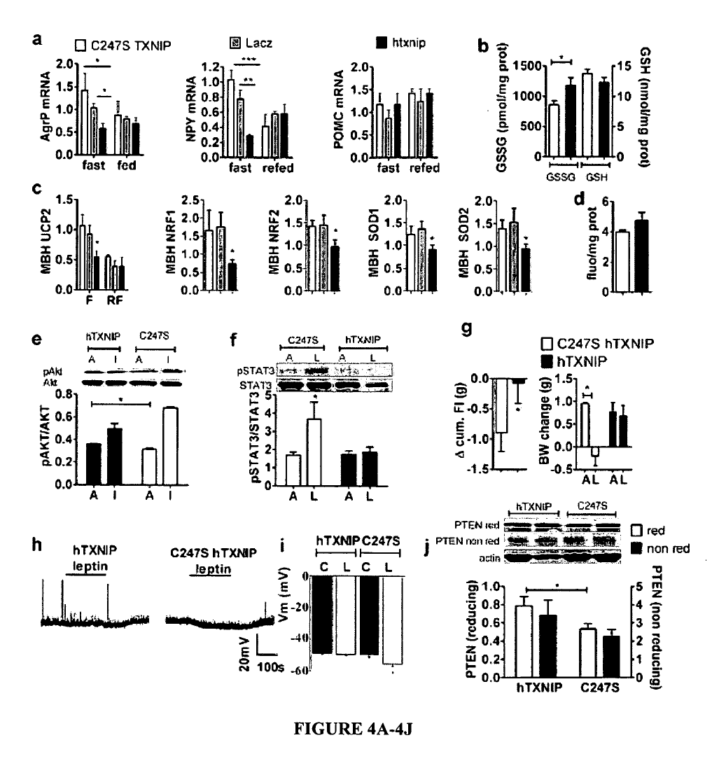

Because arcuate neurons of the melanocortin system express TXNIP and are a primary site of hypothalamic nutrient sensing, the molecular mechanisms underlying the metabolic changes induced by MBH TXNIP overexpression was assessed by measuring the expression of NPY, AgRP and POMC mRNA in the MBH. MBH expression of AgRP and NPY was lower in mice overexpressing TXNIP in the MBH compared to fasted controls, with no difference among groups in the fed state, whereas POMC expression was not affected (FIG. 4A). TXNIP overexpression and the associated decrease in thioredoxin activity likely disturbed the cellular redox state, recently identified as a contributor to hypothalamic nutrient sensing pathways (21) (22) (23), accounting for the observed changes in NPY and AgRP expression in fasted hTXNIP-expressing mice. To test this hypothesis, the intracellular redox state in MBH extracts were assessed and oxidized and reduced levels of glutathione were measured. In hTXNIP mice, oxidized glutathione levels were higher than in controls (FIG. 4B) with no change in total glutathione concentrations, indicating the induction of oxidative stress through TXNIP overexpression in the MBH. Surprisingly, the impairment in cellular redox state observed in mice expressing hTXNIP in the MBH was not accompanied by an upregulation of antioxidant pathways. Instead, it was found that increased TXNIP expression in the MBH was associated with: 1) a disruption in the fasting-induced increase in the expression of MBH UCP2 (uncoupling protein 2, a regulator of mitochondrial reactive oxygen species production (24)) with a significant decrease in UCP2 mRNA levels in mice expressing hTXNIP in the MBH compared to controls, 2) a reduction in the expression of nuclear respiratory factors NRF 1 and NRF2 that promote mitochondrial biogenesis and antioxidant responses, as well as 3) a decrease in the expression of antioxidant enzymes SOD1 and SOD2 (FIG. 4C). MBH ROS production did not differ between groups (FIG. 4D). Together, these results indicate that overexpression of TXNIP in the hypothalamus impairs the cellular redox state, mitochondrial biogenesis and antioxidant response pathways.

As hypothalamic insulin and leptin signaling are also critical to the hypothalamic control of energy homeostasis, hypothalamic insulin and leptin sensitivity were assessed in hTXNIP and C247S hTXNIP mice and the effect of parenchymal leptin and insulin infusions into the MBH on their respective signaling pathways were measured. It was found that expression of hTXNIP in the MBH impaired insulin-induced AKT phosphorylation (FIG. 4E) and leptin-induced STAT3 phosphorylation (FIG. 4F). Consistent with impaired MBH leptin signaling in mice expressing hTXNIP, intra-MBH leptin-induced anorexia and body weight loss were significantly blunted in this group compared to C247S hTXNIP controls (FIG. 4G). In addition, whole-cell patch-clamp recordings were performed from NPY neurons of the arcuate nucleus of the hypothalamus of NPY-GFP mice expressing hTXNIP or C247S hTXNIP in the MBH. It was found that MBH TXNIP overexpression blunts the electrophysiological impact of leptin on NPY neurons (FIG. 4H, 4I). Treatment with leptin (50 nM) to NPY neurons via bath application had no effect on NPY neurons in hTXNIP mice, whereas leptin significantly hyperpolarized NPY neurons in C247S hTXNIP controls (hTXNIP: −48.4±1 mV, plus leptin: −49.2±1 mV; n=13 neurons vs. C247S hTXNIP: −49.5±2 mV, plus leptin: −55.3±8 mV; n=7 neurons; p<0.05). Thus, MBH TXNIP overexpression blunts the electrophysiological, intracellular signaling and behavioral actions of MBH leptin.

Altered intracellular redox state in mice overexpressing TXNIP in the MBH could account for blunted insulin and leptin signaling through activation of phosphoprotein phosphatases such as PTEN (25). Western blot analyses of MBH from hTXNIP and C247S hTXNIP infected animals in reducing conditions revealed that PTEN expression was higher in hTXNIP mice (FIG. 4H); in non-reducing conditions, a higher level of PTEN was found in hTXNIP-infected mice than in controls, indicating higher levels of active PTEN, which opposes PI3-kinase signaling. Expression of SOCS3 in the MBH, another negative regulator of leptin signaling, was not affected. Thus, TXNIP overexpression in the MBH impairs central leptin and insulin signaling, at least in part through increased PTEN expression.

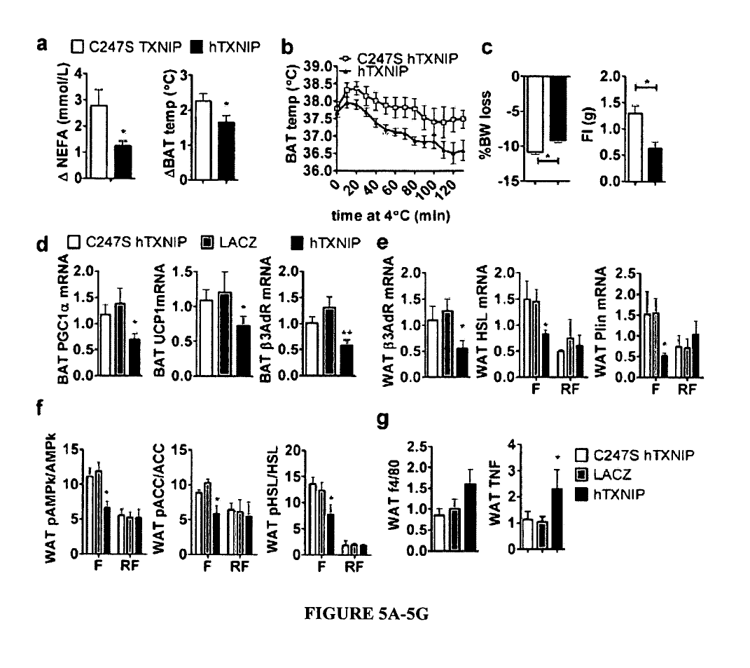

Central melanocortin signaling has been implicated in the sympathetic control of adipose tissue metabolism (4, 26-28). To test whether altered sympathetic tone resulting from decreased AgRP and NPY expression could account for disturbed lipid metabolism in mice expressing hTXNIP in the MBH, responses to metabolic challenges known to affect sympathetic activity were assessed. Intraperitoneal administration of the highly selective β3 adrenergic receptor agonist (CL 316243) activates brown fat thermogenesis and lipolysis, leading to an acute rise in circulating NEFA (29, 30). In mice expressing hTXNIP in the MBH, CL 316243-induced NEFA release and brown fat thermogenesis were lower than in C247S hTXNIP controls (FIG. 5A). Cold exposure-induced sympathetic activation, as assessed by brown fat temperature change during a cold challenge, was also decreased in hTXNIP-expressing mice (FIG. 5B). Additionally, fasting-induced lipolysis, known to be partly driven through sympathetic activation (31), was blunted in hTXNIP-expressing mice compared to C247S hTXNIP controls, as measured by the fasting-induced increase in circulating NEFA (FIG. 30), the associated weight loss, and the amount of food ingested during the refeeding phase following the fast (FIG. 5C). Together, these data indicate a decreased response to sympathetic stimuli in mice expressing hTXNIP in the MBH, which could be accounted for by decreased sympathetic tone and/or impaired adipocyte responses to sympathetic outflow. Putative mechanisms underlying the impaired brown and white fat sympathetic responses in hTXNIP mice were further evaluated decreased brown fat expression of β3 adrenergic receptor, peroxisome proliferator-activated receptor-γ coactivator (PGC)-1α and uncoupling protein 1 (UCP1) in hTXNIP mice (FIG. 5D) was found, consistent with the impaired temperature response reported above in different situations. In the epididymal adipose tissue, β3 adrenergic receptor expression was downregulated, as well as the expression of β3 adrenergic targets promoting lipolysis, such as hormone-sensitive lipase (HSL) and perilipin (Plin) (FIG. 5E), whereas caveolin and ATGL expression were not affected. Fasting-induced phosphorylation of epididymal fat AMP12 activated protein kinase (AMPK), acetylCoA carboxylase (ACC) and HSL were blunted in mice expressing hTXNIP in the MBH (FIG. 5F). Higher expression levels of TNF and F4/80 (P<0.1) in epidydimal fat of MBH hTXNIP-expressing mice indicated increased adipose tissue inflammation in this group (FIG. 5G). Last, although ACC expression was higher in hTXNIP expressing mice than in controls, expression profile of genes promoting triacylglycerol synthesis and storage in adipocytes, including lipoprotein lipase (LPL), sterol regulatory element binding protein 1c (SREBPIc), fatty acid synthase (FAS) and stearoyl-coenzyme A desaturase 1 (SCD1), were not affected. Together, these results demonstrate that TXNIP overexpression in the MBH impairs thermogenesis and lipolysis in part through alterations in the response of brown and white fat cells to sympathetic output.

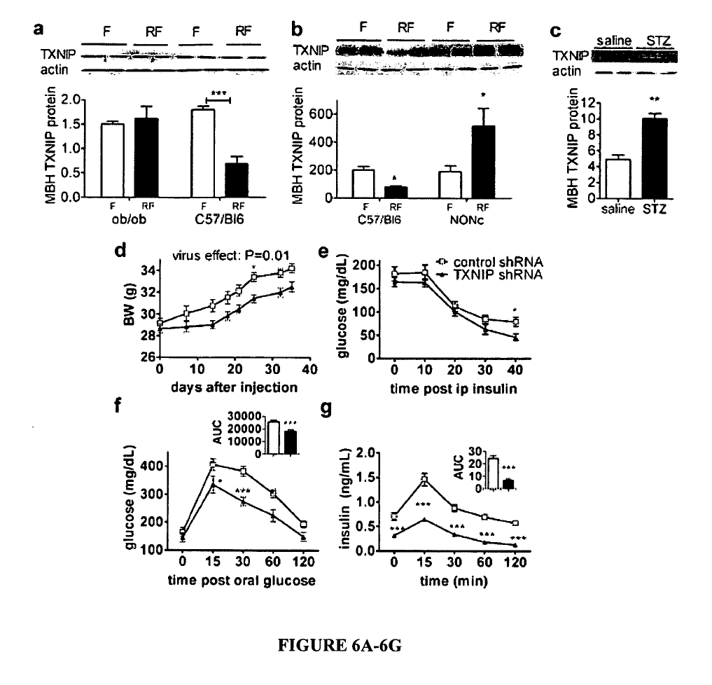

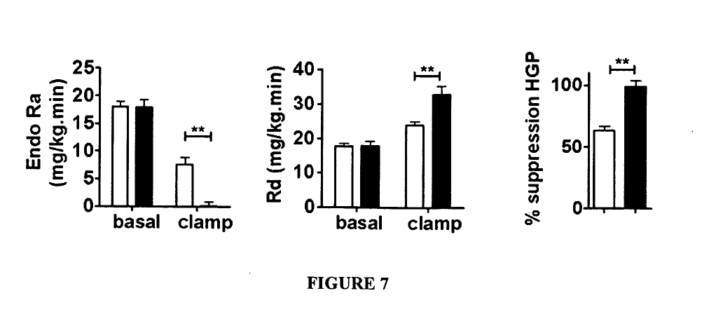

Last, the potential relevance of MBH TXNIP as a therapeutic target in the treatment of obesity and insulin resistance was tested. It was first assessed whether MBH TXNIP expression and nutritional regulation are altered in mouse models of obesity and diabetes. In leptin deficient ob/ob mice, a monogenic model of massive obesity and type 2 diabetes, TXNIP nutritional regulation in the MBH was disrupted (FIG. 6A). In 3-month old NONcNZO10/LtJ mice, a polygenic model of obesity and type 2 diabetes with adult onset diet-induced hyperglycemia and obesity, TXNIP nutritional regulation was reversed and in the fed state, MBH TXNIP expression was dramatically increased (FIG. 6B). In streptozotocin treated mice, a pharmacological model of pancreatic failure-induced hyperglycemia, a 2.5 fold increase in MBH TXNIP expression was found (FIG. 6C). Last, in mice exposed to hypothalamic glucose excess, achieved through a 4 h MBH glucose infusion, a 45% increase in TXNIP protein expression was found (TXNIP protein expression corrected to actin in the MBH: 1.71±0.20 vs. 2.39±0.28 in aCSF vs. glucose perfused mice, P<0.05, n=5). Together, these data indicate that both acute and chronic conditions of high glucose levels associated with disruptions of insulin and leptin signaling, TXNIP expression in the MBH is increased and TXNIP nutritional regulation is disrupted. To directly determine the efficiency of MBH TXNIP targeting in the prevention of obesity and insulin resistance, the MBH was targeted with lentiviruses expressing a small hairpin RNA (shRNA) against TXNIP or a control shRNA in mice fed a high-fat diet. TXNIP shRNA lentivirus functional validity was confirmed both in N41 hypothalamic cells and in MBH extracts from injected mice. MBH infection with the TXNIP shRNA led to a 40% decrease in TXNIP protein expression in the MBH and induced a 50% increase in thioredoxin activity in the MBH. Downregulation of TXNIP expression in the MBH reduced the rate of body weight gain in high fat fed mice injected with the TXNIP shRNA compared to the controls (FIG. 6D) and prevented the high-fat diet induced insulin resistance, as assessed by the glucose response to an ip. insulin challenge (FIG. 6E), and glucose intolerance (FIG. 6F, 6G). Euglycemic clamp experiments revealed that both a decrease in hepatic glucose production and an increase in peripheral glucose uptake under hyperinsulinemic conditions accounted for the improvement in glycemic control in this group compared with controls (FIG. 7). These data indicate that downregulation of MBH TXNIP expression is an effective strategy to prevent diet-induced body weight gain, fat mass deposition, and insulin resistance.

### 3. Discussion

A novel contributor to hypothalamic nutrient sensing has been identified, TXNIP, selectively expressed in neurochemically defined hypothalamic neuronal subpopulations relevant to the control of energy homeostasis. It has been shown that MBH TXNIP expression is nutritionally regulated, suppressed by anorexigenic signals (refeeding, insulin and leptin) and activated during fasting in normal physiological conditions. Importantly, in pathophysiological conditions of nutrient excess, TXNIP expression in the MBH is elevated, both acutely and in the absence of obesity and diabetes and in various mouse models of obesity and diabetes. Using a viral strategy to selectively overexpress TXNIP in the MBH, it has been demonstrated that this process affects the MBH regulation of energy balance, substrate utilization and glucose homeostasis, supporting a role for TXNIP induction in the MBH as a mechanisms by which nutrient excess affect energy homeostasis in metabolic diseases. Insight into the mechanisms linking MBH TXNIP expression and energy homeostasis has been provided, and importantly, it has been shown that MBH TXNIP expression is induced during diet-induced obesity and insulin resistance.

The increased rate of body weight gain and fat mass resulting from TXNIP overexpression in the MBH are primarily explained by a decrease in energy expenditure—evidenced by decreased oxygen consumption, physical activity and brown fat thermogenesis—and a reduction in white fat lipolytic activity in the fasted state, in the absence of any effect on feeding behavior. Two findings likely contribute to the impaired response to sympathetic challenges reported in mice overexpressing TXNIP in the MBH: 1) the observed decreases in MBH NPY/AgRP expression would attenuate melanocortinergic PVN sympathetic outflow to iBAT that controls thermogenesis (32, 28), as well as melanocortinergic sympathetic outflow to targets involved in white fat metabolism (4), and 2) the observed impairments in mediobasal hypothalamic insulin and leptin sensitivity would impede the melanocortinergic regulation of sympathetic nerve activity (33). Altered NPY tone has been associated with a shift in energy substrate utilization and a decrease in locomotor activity (34, 35), consistent with the present results. Interestingly, decreased β3-adrenergic receptor expression in both brown and white fat accompanied decreased expression and/or activity of cAMP-regulated targets such as PGC1α and its target UCP1 in the brown fat (36), and perilipin, HSL, AMPk and its target ACC in the white fat (36-38), without affecting sympathetic-independent components of lipid metabolism. Thus, decreased sympathetic activity to both brown and white adipose tissue in mice overexpressing TXNIP in the MBH represents the main phenomenon accounting for their adipose tissue metabolic phenotype.

In addition to increasing fat mass, TXNIP overexpression in the MBH also impaired glucose tolerance and insulin sensitivity. This could be secondary to adipose tissue inflammation, as evidenced by the increased epidydimal fat TNF expression, in spite of the observed decrease in circulating NEFA. Alternatively, TXNIP overexpression in the MBH could directly affect hepatic and peripheral glucose metabolism. The increased levels of plasma β-hydroxybutyrate in mice expressing hTXNIP in the MBH indeed suggests that hepatic substrate utilization is directly affected by central TXNIP expression. Central leptin and insulin resistance, a consequence of nutrient excess, lead to obesity and diabetes (39-41), and likely contribute to the impaired glycemic control in mice overexpressing TXNIP in the MBH. Together, the data identifies increased TXNIP expression in the MBH as a mechanism linking overnutrition and impaired glycemic control and energy balance.

The use of the C247S TXNIP mutant as a control demonstrates that the ability of TXNIP to bind thioredoxin is a requirement for its effect on energy homeostasis. Together with the bidirectional changes in thioredoxin activity obtained through MBH TXNIP gain and loss of function, and the measurement of an impaired intracellular redox state in mice overexpressing TXNIP in the MBH, these data support the emerging view that the redox state within hypothalamic neurons is involved in the response to nutritional signals and the regulation of energy metabolism (21-23) and involve for the first time the thioredoxin redox buffer in this regulation. It is now shown not only that its involved in acute nutrient sensing but also in the long term regulation of energy storage and utilization, an original finding. Because thioredoxin is not only a redox buffer but also a modulator of various intracellular signaling pathways through its ability to bind proteins and transcription factors (42), mechanisms alternative to redox buffering might also contribute to the metabolic phenotype of hTXNIP expressing mice. In particular, the impaired mitochondrial function, a critical component of hypothalamic nutrient sensing pathways (43, 44), decreased mitochondrial biogenesis and lower expression of mitochondrial antioxidant enzymes SOD1 and SOD2 are likely secondary to impaired thioredoxin binding to NRF1 and NRF2, as reported in other models (45). Decreased mitochondrial function in these neurons likely impairs fatty acid sensing which is known to require mitochondrial β-oxidation and UCP2 (22, 46), and this could explain why the metabolic phenotype of TXNIP-overexpressing mice manifests itself only in the context of a high-fat diet.

In summary, this work provides the first evidence in favor for a role of increased MBH TXNIP expression in obesity and diabetes as a mechanism linking nutrient excess to energy imbalance.

### REFERENCES

1. Morton G J, Cummings D E, Baskin D G, Barsh G S, Schwartz MW. Central nervous system control of food intake and body weight. Nature. 2006; 443:289-295.
2. Parton L E, et al. Glucose sensing by POMC neurons regulates glucose homeostasis and is impaired in obesity. Nature. 2007; 449:228-232.
3. Buettner C, et al. Leptin controls adipose tissue lipogenesis via central, STAT3-independent mechanisms. Nat. Med. 2008; 14:667-675.
4. Nogueiras R, et al. The central melanocortin system directly controls peripheral lipid metabolism. J Clin Invest. 2007; 117:3475-3488.
5. Nishiyama A, et al. Identification of thioredoxin-binding protein-2/vitamin D(3) up-regulated protein 1 as a negative regulator of thioredoxin function and expression. J Biol. Chem. 1999; 274:21645-21650.
6. Junn E, et al. Vitamin D3 up-regulated protein 1 mediates oxidative stress via suppressing the thioredoxin function. J. Immunol. 2000; 164:6287-6295.
7. Bodnar J S, et al. Positional cloning of the combined hyperlipidemia gene Hyplip1. Nat. Genet. 2002; 30:110-116.
8. Hui T Y, et al. Mice lacking thioredoxin-interacting protein provide evidence linking cellular redox state to appropriate response to nutritional signals. J Biol. Chem. 2004; 279:24387-24393.
9. Sheth S S, et al. Thioredoxin-interacting protein deficiency disrupts the fasting-feeding metabolic transition. J Lipid Res. 2005; 46:123-134.
10. Chen J, Saxena G, Mungrue I N, Lusis A J, Shalev A. Thioredoxin-interacting protein: a critical link between glucose toxicity and beta-cell apoptosis. Diabetes. 2008; 57:938-944.
11. Hui S T, et al. Txnip balances metabolic and growth signaling via PTEN disulfide reduction. Proc Natl Acad Sci USA. 2008; 105:3921-3926.
12. Burdakov D, Luckman S M, Verkhratsky A. Glucose-sensing neurons of the hypothalamus. Philos Trans R Soc Lond B Biol Sci. 2005; 360:2227-2235.
13. Lippoldt A, et al. Localization of thioredoxin in the rat brain and functional implications. J Neurosci. 1995; 15:6747-6756.
14. Blouet C, Jo Y H, Li X, Schwartz G J. Mediobasal hypothalamic leucine sensing regulates food intake through activation of a hypothalamus-brainstem circuit. J. Neurosci. 2009; 29:8302-8311.
15. Kitamura T, et al. Forkhead protein FoxO1 mediates Agrp-dependent effects of leptin on food intake. Nat. Med. 2006; 12:534-540.
16. de Candia P, Blekhman R, Chabot A E, Oshlack A, Gilad Y. A combination of genomic approaches reveals the role of FOXO1a in regulating an oxidative stress response pathway. PLoS ONE. 2008; 3:e1670.
17. Nakae J, et al. Regulation of insulin action and pancreatic beta-cell function by mutated alleles of the gene encoding forkhead transcription factor Foxol. Nat. Genet. 2002; 32:245-253. Epub 2002 September 2003.
18. Hosaka T, et al. Disruption of forkhead transcription factor (FOXO) family members in mice reveals their functional diversification. Proc Natl Acad Sci USA. 2004; 101:2975-2980. Epub 2004 February 2920.
19. Frescas D, Valenti L, Accili D. Nuclear trapping of the forkhead transcription factor FoxO1 via Sirt-dependent deacetylation promotes expression of glucogenetic genes. J Biol. Chem. 2005; 280:20589-20595. Epub 22005 March 20522.
20. Patwari P, Higgins L J, Chutkow W A, Yoshioka J, Lee R T. The interaction of thioredoxin with Txnip. Evidence for formation of a mixed disulfide by disulfide exchange. J Biol. Chem. 2006; 281:21884-21891.
21. Andrews Z B, et al. UCP2 mediates ghrelin's action on NPY/AgRP neurons by lowering free radicals. Nature. 2008; 454:846-851. Epub 2008 July 2030.
22. Benani A, et al. Role for mitochondrial reactive oxygen species in brain lipid sensing: redox regulation of food intake. Diabetes. 2007; 56:152-160.
23. Leloup C, et al. Mitochondrial reactive oxygen species are required for hypothalamic glucose sensing. Diabetes. 2006; 55:2084-2090.
24. Negre-Salvayre A, et al. A role for uncoupling protein-2 as a regulator of mitochondrial hydrogen peroxide generation. Faseb J. 1997; 11:809-815.
25. den Hertog J, Groen A, van der Wijk T. Redox regulation of protein-tyrosine phosphatases. Arch Biochem Biophys. 2005; 434:11-15.
26. Fan W, Morrison S F, Cao W H, Yu P. Thermogenesis activated by central melanocortin signaling is dependent on neurons in the rostral raphe pallidus (rRPa) area. Brain Res. 2007; 1179:61-69. Epub 2007 April 2006.
27. Skibicka K P, Grill H J. Energetic responses are triggered by caudal brainstem melanocortin receptor stimulation and mediated by local sympathetic effector circuits. Endocrinology. 2008; 149:3605-3616. Epub 2008 March 3627.
28. Song C K, Vaughan C H, Keen-Rhinehart E, Harris R B, Richard D, Bartness T J. Melanocortin-4 receptor mRNA expressed in sympathetic outflow neurons to brown adipose tissue: neuroanatomical and functional evidence. Am J Physiol Regul Integr Comp Physiol. 2008; 295:R417-428. Epub 2008 June 2011.
29. Grujic D, et al. Beta3-adrenergic receptors on white and brown adipocytes mediate beta3-selective agonist-induced effects on energy expenditure, insulin secretion, and food intake. A study using transgenic and gene knockout mice. J Biol. Chem. 1997; 272:17686-17693.
30. Himms-Hagen J, et al. Effect of CL-316,243, a thermogenic beta 3-agonist, on energy balance and brown and white adipose tissues in rats. Am J. Physiol. 1994; 266:R1371-1382.
31. Hucking K, Hamilton-Wessler M, Ellmerer M, Bergman R N. Burst-like control of lipolysis by the sympathetic nervous system in vivo. J Clin Invest. 2003; 111:257-264.
32. Madden C J, Morrison S F. Neurons in the paraventricular nucleus of the hypothalamus inhibit sympathetic outflow to brown adipose tissue. Am J Physiol Regul Integr Comp Physiol. 2009; 296:R831-843. Epub 2009 January 2007.
33. Rahmouni K, Haynes W G, Morgan D A, Mark A L. Role of melanocortin-4 receptors in mediating renal sympathoactivation to leptin and insulin. J. Neurosci. 2003; 23:5998-6004.
34. Currie P J, Coscina D V. Regional hypothalamic differences in neuropeptide Y-induced feeding and energy substrate utilization. Brain Res. 1996; 737:238-242.
35. Edelsbrunner M E, Herzog H, Holzer P. Evidence from knockout mice that peptide YY and neuropeptide Y enforce murine locomotion, exploration and ingestive behaviour in a circadian cycle- and gender-dependent manner. Behav Brain Res. 2009; 203:97-107. Epub 2009 May 2003.
36. Collins S, Cao W, Robidoux J. Learning new tricks from old dogs: beta-adrenergic receptors teach new lessons on firing up adipose tissue metabolism. Mol. Endocrinol. 2004; 18:2123-2131. Epub 2004 July 2128.
37. Haystead T A, Moore F, Cohen P, Hardie D G. Roles of the AMP-activated and cyclic-AMP-dependent protein kinases in the adrenaline-induced inactivation of acetyl-CoA carboxylase in rat adipocytes. Eur J. Biochem. 1990; 187:199-205.
38. Moule S K, Denton R M. The activation of p38 MAPK by the beta-adrenergic agonist isoproterenol in rat epididymal fat cells. FEBS Lett. 1998; 439:287-290.
39. Ono H, et al. Activation of hypothalamic S6 kinase mediates diet-induced hepatic insulin resistance in rats. J Clin Invest. 2008; 118:2959-2968.
40. Bence K K, et al. Neuronal PTP1B regulates body weight, adiposity and leptin action. Nat. Med. 2006; 12:917-924. Epub 2006 July 2016.
41. Kievit P, et al. Enhanced leptin sensitivity and improved glucose homeostasis in mice lacking suppressor of cytokine signaling-3 in POMC-expressing cells. Cell Metab. 2006; 4:123-132.
42. Jones D P. Radical-free biology of oxidative stress. Am J Physiol Cell Physiol. 2008; 295:C849-868. Epub 2008 August 2006.
43. Andrews Z B, Diano S, Horvath T L. Mitochondrial uncoupling proteins in the CNS: in support of function and survival. Nat Rev Neurosci. 2005; 6:829-840.
44. Obici S, Feng Z, Arduini A, Conti R, Rossetti L. Inhibition of hypothalamic carnitine palmitoyltransferase-1 decreases food intake and glucose production. Nat. Med. 2003; 9:756-761.
45. Ago T, et al. Thioredoxin 1 upregulates mitochondrial proteins related to oxidative phosphorylation and TCA cycle in the heart. Antioxid Redox Signal. 2006; 8:1635-1650.
46. Le Foll C, Irani B G, Magnan C, Dunn-Meynell A A, Levin B E. Characteristics and mechanisms of hypothalamic neuronal fatty acid sensing. Am J Physiol Regul Integr Comp Physiol. 2009; 297:R655-664. Epub 2009 June 2017.
47. van de Wall E, et al. Collective and individual functions of leptin receptor modulated neurons controlling metabolism and ingestion. Endocrinology. 2008; 149:1773-1785. Epub 2007 December 1727.
48. Azzara A V, Sokolnicki J P, Schwartz G J. Central melanocortin receptor agonist reduces spontaneous and scheduled meal size but does not augment duodenal preload-induced feeding inhibition. Physiol Behav. 2002; 77:411-416.
49. Blouet C, Ono H, Schwartz G J. Mediobasal hypothalamic p70 S6 kinase 1 modulates the control of energy homeostasis. Cell Metab. 2008; 8:459-467.
50. Holmgren A, Bjornstedt M. Thioredoxin and thioredoxin reductase. Methods Enzymol. 1995; 252:199-208.
51. Paxinos G, Franklin K B J. The Mouse Brain in Stereotaxic Coordinates, Second edition. Academic Press, NY 2001.

1. A method for determining whether an agent treats or prevents obesity and/or treats or prevents diabetes or increases insulin sensitivity, the method comprising contacting a cell which expresses thioredoxin-interacting protein (TXNIP) with the agent and measuring (i) TXNIP expression or TXNIP activity in the cell, or (ii) thioredoxin expression or activity in the cell, wherein a decrease in TXNIP expression or activity relative to a predetermined level thereof, or an increase in thioredoxin expression or activity relative to a predetermined level thereof, indicates that the agent treats or prevents obesity and/or diabetes or increases insulin sensitivity, whereas a lack of decrease in TXNIP expression or activity, or a lack of increase in thioredoxin expression or activity, indicates that the agent does not treat or prevent obesity and/or diabetes or increase insulin sensitivity.

2. The method of claim 1, wherein the method comprises measuring TXNIP expression or activity in the cell.

3. The method of claim 1, wherein the method comprises measuring thioredoxin expression or activity in the cell.

4. The method of claim 1, wherein the cell is in a fasted state.

5. The method of claim 1, wherein the predetermined level of expression or activity is determined from the cell in the absence of the agent.

6. The method of claim 1, wherein the predetermined level of expression or activity is determined from the cell in a fed state and contacted with the agent.

7. The method of claim 1, wherein the cells are mammalian.

8. The method of claim 7, wherein the cells are human.

9. The method of claim 1, wherein the cell is a hypothalamic cell or is derived from a hypothalamus.

10. (canceled)

11. A pharmaceutical composition comprising (1) an antibody directed against TXNIP which inhibits TXNIP expression or activity, an aptamer which inhibits TXNIP expression or activity, or a molecule which effects RNAi-mediated inhibition or siRNA inhibition of TXNIP expression or activity and (2) pharmaceutically acceptable carrier.

12. A method of preventing or treating obesity and/or diabetes or of increasing insulin sensitivity in a subject, the method comprising administering to the subject a therapeutically effective amount of an agent or pharmaceutical composition comprising an agent that decreases activity of, or expression of, thioredoxin-interacting protein (TXNIP).

13. The method of claim 12, wherein the agent or pharmaceutical composition decreases the activity of TXNIP.

14. The method of claim 12, wherein the subject is human.

15. The method of claim 14, wherein the agent is, or the pharmaceutical composition comprises, an antibody directed against TXNIP which inhibits TXNIP expression or activity, or an siRNA which inhibits TXNIP expression or activity.

16. The method of claim 12, wherein the agent or pharmaceutical composition is administered to the subject's central nervous system in a manner effective to reach the subject's hypothalamus.

17. A method of increasing glucose tolerance or insulin sensitivity in cells, the method comprising administering to the cells an effective amount of an agent or pharmaceutical composition comprising an agent that decreases activity of, or expression of, thioredoxin-interacting protein (TXNIP).

18. The method of claim 17, wherein the agent or pharmaceutical composition decreases the activity of TXNIP.

19. The method of claim 17 either of claim 17, wherein the cells are human.

20. The method of claim 19, wherein the agent is, or the pharmaceutical composition comprises, an antibody directed against TXNIP which inhibits TXNIP expression or activity, or an siRNA which inhibits TXNIP expression or activity.

21. The method of claim 17, wherein the cells are hypothalamic cells.

22-30. (canceled)
